(19) United States
(12) Patent Application Publication
(10) Pub. No.: US 2006/0217298 A1
(43) Pub. Date: Sep. 28, 2006

(54) Immunogenic cd91 ligand-antigenic molecule complexes and fusion proteins

(76) Inventor: Pramod K. Srivastava, Avon, CT (US)

Correspondence Address: JONES DAY, 222 EAST 41ST ST, NEW YORK, NY, 10017, US

(21) Appl. No.: 10/544,663

(22) PCT Filed: Feb. 4, 2004
(86) PCT No.: PCT/US04/03864

§ 371 Date: Mar. 6, 2006

Related U.S. Application Data
(60) Provisional application No. 60/444,766, filed on Feb. 4, 2003.

(51) Int. Cl. A61K 39/00; A61K 38/17; C07H 21/04; C12P 21/04; C12N 1/21; C07K 14/74

(52) U.S. Cl. 514/12; 530/350; 435/69.7; 435/252.3; 435/320.1; 536/23.5

(57) ABSTRACT

The present invention relates to complexes and fusion proteins comprising a CD91 ligand and an antigenic molecule, for use in the treatment or prevention of a disease. The invention specifically provides complexes comprising a CD91 ligand non-covalently bound to, or alternatively crosslinked to, an antigenic molecule. The invention also specifically provides fusion proteins comprising a CD91 ligand fused via a peptide bond to an antigenic molecule.

This application claims the benefit of U.S. Provisional Application No. 60/444,766, filed on Feb. 4, 2003, which is incorporated by reference herein in its entirety.

This invention was made with government support under Grant No. CA/A184479 awarded by the National Institutes of Health. The government has certain rights in the invention.

### 1. INTRODUCTION

The present invention relates to complexes and fusion proteins comprising a CD91 ligand and an antigenic molecule, for use in the treatment or prevention of a disease.

### 2. BACKGROUND OF THE INVENTION

CD91, also known as LDL receptor related protein, is structurally related to other receptors in the LDL receptor gene family. Like other members of the LDL receptor gene family, CD91 has various functions besides lipid metabolism, including homeostasis of proteinases and proteinase inhibitors, cellular entry of toxins and viruses, lysosomal enzyme activation, cellular signal transduction and neurotransmission. Herz and Srtickland, 2001, J. Clin. Invest. 108(6):779-84.

CD91 is primarily expressed in liver, brain and placenta The extracellular domain of the human receptor comprises six 50-amino acid EGF repeats and 31 complement repeats of approximately 40-42 amino acids. The complement repeats are organized, from the amino to the carboxy-terminus, into clusters of 2, 8, 10 and 11 repeats, called Cluster I, II, III and IV (Herz et al., 1988, EMBO J. 7:4119-4127). One study points to Cluster II (Cl-II), which contains complement repeats 3-10 (CR3-10), as the major ligand binding portion of the receptor (Horn et al., 1997, J. Biol. Chem. 272:13608-13613).

CD91 binds to at least 30 different ligands with high affinity, including ApoE, lipoprotein lipase, hepatic lipase, factor IXa, factor VIIIa, factor VIIa/TFPI, MMP-13 (matrix metaloproteinase-13), MMP-9 (matrix metaloproteinase-9), spingolipid activator protein, pregnancy zone protein, PAI-1 (plasminogen activator inhibitor-1), antithrombin III, tissue factor pathway inhibitor (TFPI), Pseudomonas exotoxin A, heparin cofactor II, APP (amyloid precursor protein), thrombospondin-1, thrombospondin-2, lactoferrin, rhinovirus, RAP (alpha-2 macroglobulin receptor associated protein) and HIV-Tat protein. A description of CD91 and CD91 ligands is provided by Herz and Strickland, 2001, J. Clin. Invest. 108:779-784, which is incorporated by reference herein.

The receptor binding domains of some CD91 ligands have been identified and are listed in section 4.3.

### 3. SUMMARY OF THE INVENTION

The present invention relates to novel complexes and fusion proteins comprising a CD91 ligand and an antigenic molecule. The complexes and fusion proteins of the invention can induce an immune response against the antigenic molecule and are useful in the treatment or prevention of a disease. The invention also relates to methods of producing the complexes and fusion proteins, as well as compositions of complexes or fusion proteins.

The present invention also provides methods of treating or preventing a disease in a subject using said complexes and fusion proteins and methods of inducing an immune response in a subject using said complexes and fusion proteins.

The following CD91 ligands, listed by way of example and not limitation, are suitable for use in the invention: ApoE, lipoprotein lipase, hepatic lipase, factor IXa, factor VIIIa, factor VIIa/TFPI, MNO-13, MMP-9, spingolipid activator protein, pregnancy zone protein, PAI-1, antithrombin E, Pseudomonas exotoxin A, tissue factor pathway inhibitor (ITPI), heparin cofactor II, APP, thrombospondin-1, thrombospondin-2, lactoferrin, rhinovirus, RAP and HIV-Tat protein.

In one aspect of the invention, a purified complex is provided comprising a CD91 ligand and an antigenic molecule. In one embodiment, the complex is immunogenic, i.e., when the complex is introduced into a suitable host, the host develops an immune response against the antigenic molecule of the complex. In another embodiment, the antigenic molecule is not an antibody or a derivative thereof comprising a binding region thereof. In one embodiment, the CD91 ligand is noncovalently associated with the antigenic molecule. In another embodiment, the CD91 ligand is cross-linked to the antigenic molecule. In yet another embodiment, the complex is the product of a method comprising complexing said CD91 ligand and said antigenic molecule in vitro. In another embodiment, the antigenic molecule is a protein. In a further embodiment, the antigenic molecule is derived from an antigenic cell. In another embodiment, the complex is free of biological cells and vesicles. In another embodiment, the CD91 ligand is derived from a mammal. In a further embodiment, the CD91 ligand is derived from a human. In another embodiment, the antigenic molecule is derived from a mammal. In another embodiment the antigenic molecule is derived from a human. In a yet a further embodiment the CD91 ligand and the antigenic molecule are derived from the same subject. In another embodiment, the CD91 ligand and the antigenic molecule are derived from different subjects.

In another aspect of the invention, a purified complex is provided comprising a fragment of a CD91 ligand and an antigenic molecule. In a particular embodiment, the complex is immunogenic. In a preferred embodiment, the fragment comprises a receptor binding domain.

In another embodiment of the invention, a purified fusion protein is provided comprising an antigenic protein fused via a peptide bond to a CD91 ligand. In another embodiment, the fusion protein is immunogenic. In a further embodiment, the complex is free of biological cells and vesicles.

In another embodiment of the invention, a purified fusion protein is provided comprising an antigenic protein fused via a peptide bond to a fragment of a CD91 ligand. In a particular embodiment, the fusion protein is immunogenic. In another embodiment, the antigenic protein is not an antibody or a part thereof. In a preferred embodiment, the fragment comprises a receptor binding domain.

In another embodiment, the invention provides a composition comprising a plurality of purified complexes. In a further embodiment, each complex comprises a different antigenic molecule. In another embodiment, the population of complexes of CD91 ligands bound to antigenic molecules is purified to apparent homogeneity, as viewed on an SDS-PAGE gel.

In another aspect of the invention, a pharmaceutical composition is provided comprising an amount of purified complex effective for treatment or prevention of a disease or a disorder. In a particular embodiment, the complex of the pharmaceutical composition is immunogenic. In another embodiment, the pharmaceutical composition further comprises a pharmaceutically acceptable carrier.

Another embodiment of the invention provides a method for preparing immunogenic, in vitro complexes of a CD91 ligand associated with one or more antigenic molecules. In one embodiment, the method comprises a) incubating a CD91 ligand or fragment thereof and one or more antigenic molecules under conditions and for a length of time sufficient for formation of complexes of the CD91 ligand non-covalently bound to the antigenic molecules; and b) isolating said complexes. In a further embodiment of this method, the complexes are free of biological cells and vesicles. In yet a further embodiment, the CD91 ligand is purified.

In a further aspect of the invention, a method of treating or preventing a disease in a subject is provided. In one embodiment, the method comprises administering to the subject an amount of a purified complex effective to treat or prevent a disease. In a further embodiment, the CD91 ligand is noncovalently associated with the antigenic molecule. In another embodiment, the CD91 ligand is cross-linked to the antigenic molecule. In another aspect of the invention, the complex is free of biological cells and vesicles. In yet a further embodiment, the antigenic molecule displays the antigenicity of a cancer-associated or cancer-specific antigen, or of an antigen that causes or is associated with a disease, respectively.

Another embodiment of the invention provides a method of treating or preventing a disease in a subject comprising administering to the subject an amount of a purified fusion protein comprising an antigenic protein fused via a peptide bond to a CD91 ligand effective to treat or prevent a disease. In a further embodiment, the fusion protein is free of biological cells and vesicles. In another embodiment, the antigenic molecule displays the antigenicity of a cancer-associated or cancer-specific antigen, or of an antigen that causes or is associated with a disease, respectively.

The invention further provides a recombinant cell transformed with a) a first nucleic acid comprising a first nucleotide sequence that is operably linked to a first promoter and that encodes a CD91 ligand, and b) a second nucleic acid comprising a second nucleotide sequence that is operably linked to a second promoter and encodes an antigenic molecule, such that the CD91 ligand and the antigenic molecule are expressed within the cell and non-covalently associate with each other to form a complex that in sufficient amount is capable of eliciting an immune response to the antigenic molecule. In one embodiment, the cell is a human cell.

In another aspect of the invention, a recombinant cell is provided that is transformed with a nucleic acid comprising a nucleotide sequence that is operably linked to a promoter, wherein said nucleotide sequence encodes a fusion protein comprising an antigenic protein fused via a peptide bond to a CD91 ligand. In a particular embodiment, the fusion protein is immunogenic.

In one embodiment, the invention provides pharmaceutical compositions comprising recombinant cells and pharmaceutically acceptable carriers.

In another aspect of the invention, a method for preparing in vitro complexes of a CD91 ligand associated with one or more antigenic molecules comprising cross-linking the CD91 ligand to one or more antigenic molecules using a cross-linking agent is provided. In another embodiment,.the complex is immunogenic.

Another aspect of the invention provides a method for preparing a fusion protein comprising a) culturing a recombinant cell transformed with a nucleic acid comprising a nucleotide sequence that is operably linked to a promoter, wherein said nucleotide sequence encodes a fusion protein comprising an antigenic protein fused via a peptide bond to a CD91 ligand, under conditions such that the fusion protein is expressed by the cell and b) recovering the fusion protein from the cells. In a particular embodiment, the fusion protein is immunogenic.

In another embodiment of the invention, the complexes of the invention can be used in combination with other therapies in the treatment or prevention of a disease or disorder.

Another aspect of the invention provides a method of inducing an immune response against an antigenic molecule in a subject comprising administering to the subject an effective amount of a purified complex comprising said antigenic molecule and a CD91 ligand. In one embodiment, the CD91 ligand and antigenic molecule are non-covalently bound to each other. In another embodiment, the CD91 ligand and antigenic molecule are cross-linked to each other. In another embodiment, the complex is free of biological cells and vesicles. In another embodiment, the immune response to the antigen is induced by enhancing uptake of said antigenic molecule by antigen presenting cells.

Another aspect of the invention provides a method of inducing an immune response against an antigenic protein in a subject comprising administering to the subject an effective amount of purified fusion protein comprising said antigenic protein fused via a peptide bond to a CD91 ligand. In another embodiment, the fusion protein is free of biological cells and vesicles. In another embodiment, the immune response is induced by enhancing uptake of said antigenic protein by antigen presenting cells.

### 4. DETAILED DESCRIPTION OF THE INVENTION

The present-invention provides complexes and fusion proteins of CD91 ligands and antigenic molecules, compositions of said fusion proteins and complexes, methods of preparing said fusion proteins and complexes, methods of treating or preventing a disease in a subject by immunizing with said fusion proteins and complexes, and methods of inducing an immune response to an antigenic molecule using the complexes and fusion proteins of the invention.

In certain embodiments, the compositions and formulations of the present invention are administered to a subject to prevent a disease, including inhibiting the onset or development of the disease in a subject not having the disease, and inhibiting the progression of a disease in an asymptomatic subject. Preferably, the complexes and or fusion proteins of the invention in such compositions and formulations are purified.

The term “disease” means any abnormal physical or mental condition, or a condition of a living animal or plant body or of one of its parts that impairs normal functioning. Types of disease include, but are not limited to cancer, infectious diseases, neurodegenerative diseases, endocrine/netabolic diseases and vascular diseases.

In other embodiments, the compositions and formulations of the present invention are administered to a subject that has been diagnosed with a disease or is suspected of having a disease. According to the present invention, treatment of a disease encompasses the treatment of subjects already diagnosed as having a disease; the delay of the onset or evolution or aggravation or deterioration of the symptoms or signs of a disease; and/or promoting regression of a disease in symptomatic subjects.

The term “CD91 ligand” means a molecule that binds to CD91.

The term “antigenic molecule” means any molecule containing an antigenic determinant, i.e., capable of being bound by an antibody or recognized by a T cell in the context of MHC Class I, Class II molecules, or CD1 molecules. Antigenic molecules can be, but not limited to, carbohydrates, lipids, polypeptides and peptides (polypeptides and peptides collectively referred to as “proteins”). The term “antigenic molecule” does not encompass an antibody or a derivative thereof comprising a binding region thereof In one embodiment, an antigenic molecule comprises one or more antigenic determinants (epitopes) of a single protein. In another embodiment, an antigenic molecule comprises multiple antigenic determinants, at least one antigenic determinant from each of two or more proteins. In another embodiment, an antigenic molecule comprises one or more antigenic determinants (epitopes) of a single naturally occurring protein. In another embodiment, an antigenic molecule comprises multiple antigenic determinants, at least one antigenic determinant from each of two or more naturally occuring proteins. In another embodiment, the antigenic molecule comprises one or more antigenic determinants from a first naturally occurring protein and one or more antigenic determinants from a second naturally occurring protein, i.e., it can be a fusion protein of at least a portion of two different naturally occurring proteins.

The term “immunogenic” in reference to a substance means that the substance, when introduced into a suitable host, can cause the production by the host of an immune response (antibody or cell-mediated) against the substance. * Complexes between CD91 ligands and antigenic molecules can be produced by any of a number of methods. For example, antigenic molecules associated with a disease can be obtained by recombinant or synthetic methods, or can be isolated and purified from recombinant cells. The antigenic molecules can also be isolated from cells removed from a subject or cells in culture. Complexes between antigenic molecules and CD91 ligands can be formed by covalent or non-covalent association of antigenic molecules with CD91 ligands. Preferably, the complexes of the invention are used in purified form, preferably to apparent homogeneity as viewed on an SDS-PAGE gel, or to at least 60%, 70%, 80%, or 90% of total protein.

In addition, complexes may be formed in vitro using a variety of methods, described herein. Methods for preparing such CD91 ligand-antigenic molecule complexes are described in detail in Sections 4.3 to 4.5, below.

CD91 ligand-antigenic peptide complexes and fusion proteins may be used as vaccines against various diseases. Without being bound by any particular theory, such complexes or fusion proteins may act by eliciting a B-cell and/or T-cell response in patients with such disorders. Methods for the use of such CD91 ligand-antigenic molecule complexes and fusion proteins as vaccines against various diseases are described in Section 4.6 in detail herein.

4.1 Sources of Antigenic Molecules

According to the invention, the complexes comprise antigenic molecules complexed to CD91 ligands. In one embodiment, the antigenic molecule is prepared by synthetic means. In another embodiment the antigenic molecule is prepared by recombinant methods. In yet another embodiment, the antigenic molecules are from a preparation of proteins from an antigenic cell of interest. In yet another embodiment, the antigenic molecules are prepared from tissue which displays the pathologic or histologic changes associated with a disease. In another embodiment of the invention, the antigenic molecules are prepared from body fluid or any other biological source.

The compositions of the invention also comprise complexes of CD91 ligands and antigenic peptides that are prepared by first, generating a population of peptides from a preparation of proteins of the antigenic cells of interest, and then complexing the peptides to CD91 ligands.

In various specific embodiments, where it is desired to maximize and preserve the diversity of antigenic proteins and peptides for use as antigenic molecules in the complexes 5 of the invention, the methods used for preparing a protein preparation of antigenic cells do not selectively remove or retain any particular protein or peptide from other proteins and peptides in the antigenic cell. Even in certain embodiments when cytosolic proteins or membrane-derived proteins are used, the methods used to make the preparations do not selectively remove or retain any particular protein of the cytosol or of the membranes. Therefore, the majority of the proteins present in the cytosol or in the membranes are also present in the respective preparations of antigenic proteins and peptides from antigenic cells. In preferred embodiments, substantially the entire repertoire of antigenic proteins and peptides of the antigenic cells, and substantially all the antigenic proteins and peptides in the cytosol or in the membranes are present in the complexing reaction and form complexes with CD91 ligands.

Antigenic proteins or antigenic fragments thereof may be used as antigenic molecules. Optionally, the proteins or fragments may be purified. Antigenic epitopes of proteins may optionally be screened using any method known in the art. Such techniques include, but are not limited to, methods that are based on algorithmic identification, peptide elution and cell-based binding assay techniques. Protein sequences may be analyzed to identify antigen-specific epitopes that meet criteria such as conservancy, binding, population coverage and immunogenicity. Potential epitopes can be identified by, e.g. a hydrophilicity analysis (see e.g., Hopp and Woods, 1981, Proc. Natl. Acad. Sci. USA 78:3824).

Another such process for epitope identification is described in Sette et al., 2002, Curr Opin Investig Drugs 3:132-9. Briefly, computer algorithms analyze the amino acid sequence of all known antigens associated with the target indication for the presence of peptides which contain epitope motifs and which meet sequence conservancy requirements (epitopes from variable regions of the antigen are avoided). Peptides meeting the requirements of the computer screen are synthesized and in vitro HILA binding assays are performed. Peptides are evaluated for superfamily binding (assessing their ability to bind broadly within a family of HLA molecules). Identifying epitopes which bind broadly within superfamilies ensures broad population coverage for the ultimate vaccine. Peptides are then tested for immunogenicity, both in vivo in mice which express human MAC and in vitro against infected or transfected cells.

Using such screening techniques allows identification of the specific amino acid sequence or sequences of a protein which elicit an immune response. Disease relevant epitopes maybe tested and validated via functional T-cell assays, ensuring their clinical relevance, e.g., screened using T-cells from humans, confronted with the disease and who developed a protective natural immune response. Epitopes may be modified, e.g., by changing one or more amino acid residues, to enhance/optimize immunogenicity. See e.g., U.S. Pat. No. 6,037,135, herein expressly incorporated by reference in its entirety. The invention provides complexes comprising these antigenic epitopes and CD91 ligands.

In a specific embodiment of the invention, where it is desired to treat or prevent cancer, a tumor specific or tumor associated antigen maybe used as the antigenic molecule in the complexes or fusion proteins of the invention. Any such antigens known in the art may be used in the complexes and fusion proteins of the invention, in addition to the following tumor antigens, listed by way of example and not limitation: 707-AP (707 alanine proline), AFP (alpha (α)-fetoprotein), ART-4 (adenocarcinoma antigen recognized by T cells 4), BAGE (B antigen; β-catenin/m, β-catenin/mutated), Ber-ab1 (breakpoint cluster region-Abelson), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis antigen), Cyp-B (cyclophilin B), DAM (differentiation antigen melanoma), ELF2M (elongation factor 2 mutated), ETV6-AML1 (Ets variant gene 6/acute myeloid leukemia 1 gene ETS), G250 (glycoprotein 250), GAGE (G antigen), GnT-V (N-acetylglucosaminyltransferase V), Gp100 (glycoprotein 100 kD), HAGE (helicose antigen), HER-2/neu (human epidermal receptor-2/neurological), HLA-A *020119 R170I (arginine (R) to isoleucine (I) exchange at residue 170 of the α-helix of the α2-domain in the HLA-A2 gene), HPV-E7 (human papilloma virus E7), HSP70-2M (heat shock protein 70-2 mutated), HST-2 (human signet ring tumor-2), hTERT or hTRT (human telomerase reverse transcriptase), iCE (intestinal carboxyl esterase), KIAA0205, LAGE (L antigen), LDLR/FUT (low density lipid receptor/GDP-L-fucose), β-D-galactosidase 2-α-L-fucosyltransferase, MAGE (melanoma antigen), MART-1/Melan-A (melanoma antigen recognized by T cells-1/Melanoma antigen A), MC1R melanocortin 1 receptor, Myosin/m (myosin mutated), MUC1 (mucin 1 ), MUM-1, -2, -3 (melanoma ubiquitous mutated 1, 2, 3), NA88-A (NA cDNA clone of patient M88), NY-ESO-1 (New York-esophagus 1), P15 (protein 15), p190 minor brr-ab1 (protein of 190 KD bcr-ab1), Pm1/RARα (promyelocytic leukaemialretinoic acid receptor α), PRAME (preferentially expressed antigen of melanoma), PSA (prostate-specific antigen), PSM (prostate-specific membrane antigen), RAGE (renal antigen RU1 or RU2 or renal ubiquitous 1 or 2), SAGE (sarcoma antigen), SART-1 or SART-3 (squamous antigen rejecting tumor 1 or 3), TEL/AMLI (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2) and WT1 (Wilms' tumor gene).

In a specific embodiment of the invention, where it is desired to treat or prevent an infectious disease, a molecule comprising one or more epitopes of an infectious agent (e.g., viral antigen, bacterial antigen, etc.) that is the causative agent of the disease is used. Preferably, where it is desired to treat or prevent a viral disease, a molecule comprising epitope(s) of a virus is used; where it is desired to treat or prevent a bacterial infection, a molecule comprising epitope(s) of bacteria is used; where it is desired to treat or prevent protozoal infection, a molecule comprising epitope(s) of protozoa is used; and where it is desired to treat or prevent a parasitic infection, a molecule comprising epitope(s) of parasites is used.

Preferably, where it is desired to treat or prevent a neurodegenerative or amyloid disease, a molecule comprising epitope(s) of an antigenic molecule associated with a neurodegenerative disease, or epitope(s) of an antigenic molecule associated with an amyloid disease, including but not limited to a fibril peptide or protein, is used as the antigenic molecule of the invention. For example, such a neurodegenerative disease-associated antigenic molecules may be a molecule associated with Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, spongiform encephalopathies, Creutzfeldt-Jakob disease, polyglutamine diseases, Huntington's disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder. Antigenic molecules that are suitable for in vitro complexing methods are disclosed in PCT publication no. WO 01/52890, dated Jul. 26, 2001, which is incorporated by reference herein in its entirety, and include, but are not limited to, β-amyloid or a fragment thereof an oligomeric Aβ complex or a fragment thereof, an ApoE4-Aβ complex, tau protein or an fragment thereof, amyloid precursor protein or a fragment thereof, a mutant amyloid precursor protein or a fragment thereof, presenillin or a fragment thereof, a mutant of presenillin or a fragment thereof, α-synuclein or a fragment thereof, or a prion protein or a fragment thereof, and the antigenic derivatives of any of the foregoing proteins or fragments thereof Amyloid disease associated antigenic molecules may be molecules associated with diseases characterized by the extracellular deposition of protein and/or peptide fibrils which form amyloid deposits or plaques, including but not limited to type II diabetes and amyloidoses associated with chronic inflammatory or infectious disease states and malignant neoplasms, e.g., myeloma. Certain amyloid disease such as Alzheimer's disease and prion diseases, e.g., Creutzfeldt Jacob disease, are neurodegenerative diseases.

In another embodiment, for the treatment or prevention of an autoimmune disease, the antigenic molecule of the invention is related to an autoimmune disease. These autoimmune diseases include, but are not limited to, insulin-dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, buflous pemphigoid, discoid lupus, ulcerative colitis, and dense deposit disease. Thus, for example, a cytokine can be an antigenic molecule.

Preferably, where it is desired to treat or prevent an endocrine or metabolic disease, molecules comprising epitopes of antigenic molecules associated with endocrine or metabolic diseases are used as the antigenic molecules of the invention. Thus, for example, cholesteryl ester transfer protein can be an antigenic molecule.

Preferably, where it is desired to treat or prevent vascular diseases (e.g., cardiovascular disease), molecules comprising epitopes of antigenic molecules associated with vascular diseases are used as the antigenic molecules of the invention. Thus, for example, angiotensin II can be an antigenic molecule in the treatment or prevention of vascular diseases.

4.1.1 Preparations of Antigenic Molecules From Antigenic Cells of Interest

Antigenic proteins used in the complexes of the invention can be obtained from antigenic cells of interest.

Since whole cancer cells, infected cells or other antigenic cells are used in one embodiment of the present methods, in such embodiment, it is not necessary to isolate or characterize or even know the identities of these antigenic proteins in advance of using such methods. The source of the antigenic cells may be selected, depending on the nature of the disease with which the antigens are associated. In one embodiment of the invention, any tissues, or cells isolated from a cancer, including cancer that has metastasized to multiple sites, can be used as an antigenic cell in the present method. For example, leukemic cells circulating in blood, lymph or other body fluids can also be used, solid tumor tissue (e.g., primary tissue from a biopsy) can be used. In a specific embodiment, antigenic cells can be cancer cells or preneoplastic cells. The transition from non-neoplastic cell growth to neoplasia commonly consists of hyperplasia, metaplasia, and dysplasia (for review of such abnormal growth conditions (See Robbins and Angell, 1976, Basic Pathology, 2d Ed., W.B. Saunders Co., Philadelphia, pp. 68-79). A non-limiting list of cancers, the cells of which can be used herein is provided in Section 4.13.

Cell lines derived from cancer tissues, cancer cells, infected cells, or cells that displays the pathologic or histologic changes associated with a disease can also be used as antigenic cells. Cancer or infected tissues, cells, or cell lines of human origin are preferred. Cancer cells, infected cells, or antigenic cells can be identified and isolated by any method known in the art. For example, cancer cells or infected cells can be identified by morphology, enzyme assays, proliferation assays, or the presence of pathogens or cancer-causing viruses. If the characteristics of the antigenic molecule of interest are known, antigenic cells can also be identified or isolated by any biochemical or immunological methods known in the art. For example, cancer cells or infected cells can be isolated by surgery, endoscopy, other biopsy techniques, affinity chromatography, and fluorescence activated cell sorting (e.g. with fluorescently tagged antibody against an antigen express by the cells). Antigenic cells that display similar antigenicity have one or more antigenic determinants in common against which an immune response in a subject is desired (e.g., for therapeutic or prophylactic purposes).

If the number of antigenic cells obtained from a subject is insufficient, the cells may be cultured in vitro by standard methods to expand the number of cells prior to use in the present methods. There is no requirement that a clonal or homogeneous or purified population of antigenic cells be used. A mixture of cells can be used provided that a substantial number of cells in the mixture contain the antigens of interest. In a specific embodiment, the antigenic cells and/or immune cells are purified.

For the treatment or prevention of a type of cancer, the methods of the invention provide compositions of a CD91 ligand complexed to an antigenic molecule displaying the antigenicity of a tumor or cancer antigen of the same type of cancer, e.g., an antigen overexpressed in tumor or cancer cells relative to nontumor or noncancerous cells (“tumor associated antigens”), or an antigen expressed in tumor or cancer cells and not expressed in nontumor or noncancerous cells (“tumor specific antigen”). As used herein, the term “the same type of cancer” refers to cancer of the same tissue type, or metastasized from cancer of the same tissue type. In one embodiment, the antigenic molecules are antigenic peptides derived from cancer cells, preferably human cancers, e.g., tumor specific antigens and tumor associated antigens. The peptides can be generated by proteolytic digestion of proteins (e.g., cytosolic and/or membrane-derived proteins) from cancer cells, or antigenic cells that share antigenic determinants with or display similar antigenicity as the cancer cells.

In another embodiment, the antigenic molecules are antigenic peptides derived from cells infected by a pathogen or infectious agent that causes the infectious disease, or the pathogen which includes but is not limited to, a virus, bacterium, fungus, protozoan, parasite, etc. Preferably, the pathogen is one that infects humans. For example, for the treatment or prevention of infectious diseases, the methods of the invention provide compositions of CD91 ligands complexed to antigenic molecules that display the antigenicity of an antigen of an infectious agent that causes an infectious disease or of an antigen that is associated with or causes an infectious disease. The antigenic peptides are generated by proteolytic digestion of (e.g., cytosolic and/or membrane-derived) proteins obtained from infected cells, antigenic cells that share antigenic determinants with or display similar antigenicity as the infected cells, or the pathogens including viral particles. The antigenic peptides can also be generated from antigenic cells that display the antigenicity of an agent (pathogen) that causes the infectious disease, or a variant of such agent. Infectious agents that can infect cells which can be used herein is provided in Section 4.14.

In yet another embodiment, any pathogen or infectious agent that can cause an infectious disease can be used to infect a cell, and the infected cell used as an antigenic cell for the preparation of antigenic peptides. Variants of a pathogen or infectious agent, such as but limited to replication-defective variants, non-pathogenic or attenuated variants, non-infectious variants, can also be used as an antigenic cell for this purpose. For example, many viruses, bacteria, fungi, parasites and protozoans that can be cultured in vitro or isolated from infected materials can serve as a source of antigenic cells. Methods known in the art for propagating such pathogens including viral particles can be used.

In order to prepare pathogen-infected cells, uninfected cells of a cell type susceptible to infection by the pathogen or infectious agent that causes the disease can be infected in vitro. Depending on the mode of transmission and the biology of the pathogen or infectious agent, standard techniques can be used to facilitate infection by the pathogen or infectious agent, and propagation of the infected cells. For example, influenza viruses may be used to infect normal human fibroblasts; and mycobacteria may be used to infect normal human Schwann cells. In various embodiments, variants of an infectious agent, such as replication-defective viruses, non-pathogenic or attenuated mutants, or temperature-sensitive mutants can also be used to infect or transform cells to generate antigenic cells for the preparation of antigenic peptides. If large numbers of a pathogen are needed to infect cells, or if pathogens are used directly as antigenic cells, any method known in the art can be used to propagate and grow the pathogens. Such methods will depend on the pathogen, and may not involve infecting a host. For example, many techniques are known in the art for growing pathogenic bacteria, fungi and other non-viral microorganisms in culture, including large scale fermentation.

In another embodiment, any cell or tissue that displays the pathologic or histologic changes associated with a disease can be used as antigenic cell for the preparation of antigenic peptides. For example, neurons that display the pathologic changes associated with Alzheimer's disease, such as plaque formation, are suitable as antigenic cells.

In another embodiment of the invention for the treatment or prevention of a disease, the methods of the invention provide compositions of CD91 ligands complexed to antigenic molecules displaying the antigenicity of an antigen that causes or is associated with the disease. In a specific embodiment, the antigenic molecules are from cells which display the pathologic or histologic changes associated with the disease.

In a specific embodiment of the invention, if a gene encoding an antigenic protein is available, normal cells of the appropriate cell type from the intended recipient may be transformed or transfected in vitro with an expression construct comprising a nucleic acid molecule encoding such antigen, such that the antigen is expressed in the recipient's cells.

Optionally, more than one such antigen may be expressed in the recipient's cell in this fashion, as will be appreciated by those skilled in the art, any techniques known, such as those described in Ausubel et al., 1989, Current Protocols in Molecular Biology, Greene Publishing Associates and Wiley lnterscience, New York, may be used to perform the transformation or transfection and subsequent recombinant expression of the antigen gene in recipient's cells.

In one embodiment of the invention, a protein preparation is provided which is derived from a pathogen, a cancer cell, an infected cell or a cell that displays the pathologic or histologic changes associated with a disease. For example, for the treatment of cancer, the protein preparations are prepared, postoperatively, from tumor cells obtained from a cancer patient. In another embodiment of the present invention, one or more antigenic proteins of interest are synthesized in cell lines modified by the introduction of recombinant expression systems that encode such antigens, and such cells are used to prepare the proteins. The proteins can be obtained from one or more cellular fraction(s), for example, the cytosol of the antigenic cells, or they can be extracted or solubilized from the membranes or cell walls of the antigenic cells. Any technique known in the art for cell lysis, fractionation of cellular contents, and protein enrichment or isolation can be used. See, for example, Current Protocols in Immunology, vol. 2, chapter 8, Coligan et al. (ed.), John Wiley & Sons, Inc.; Pathogenic and Clinical Microbiology: A Laboratory Manual by Rowland et al., Little Brown & Co., June 1994; which are incorporated herein by reference in their entireties. Depending on the techniques used to fractionate the cellular contents, a cellular fraction comprises at least 20, 50, 100, 500, 1,000, 5,000, 10,000, or 20,000 different proteins.

As used herein, the term “protein preparation” refers to a mixture of proteins obtained from antigenic cells, a cellular fraction of antigenic cells, or virus particles. The proteins can be obtained from a cellular fraction, such as the cytosol. The proteins can also be non-cytosolic proteins (e.g., those from cell walls, cell membranes or organelles), or both Cellular fractions may include but are not limited to cytosolic fractions, membrane fractions, and organelle fractions, such as nuclear, mitochondrial, lysosomal, and endoplasmic reticulum-derived fractions. The protein preparations can be obtained from non-recombinant or recombinant cells. The term “antigenic proteins” as used herein also encompasses antigenic polypeptides and antigenic peptides that may be present in the preparation The protein preparation obtained from the antigenic cells or cellular fractions thereof or virus particles can optionally be purified from other non-proteinaceous materials to various degrees by techniques known in the art. The protein preparation may comprise at least 50%, 55%, 60%, 65%, 70%, 75%, 80%, 85%, 90%, 95% 97%, 98%, 99% of the different proteins and peptides present in the antigenic cells or virus particles or a fraction of the antigenic cells.

In a specific embodiment, the protein preparations have not been subjected to any method of preparation that selectively removes or retains one or more particular protein(s) from the other proteins in the antigenic cells.

In a specific embodiment, the protein preparation is the total cell lysate which is not fractionated and/or purified, and may contain other non-proteinaceous materials of the cells. In another specific embodiment, the protein preparation is total protein in a cellular fraction, which has not been subjected to frdher fractionation or purification, and may contain other non-proteinaceous materials of the cells. In yet another embodiment, the protein preparation is the total protein in a preparation of viral particles. In specific embodiments, the protein preparation comprises total cellular protein, total cytosolic proteins, or total membrane-bound proteins of antigenic cell(s). In various embodiments, the protein preparation comprises at least 20, 50, 100, 500, 1,000, 5,000, 10,000, or 20,000 different proteins. A plurality of different antigenic proteins are present in a protein preparation of antigenic cells. Moreover, the proteins in the protein preparation may be subjected to a step of protease digestion prior to in vitro complexing to CD91 ligands. Alternatively, the proteins in the protein preparation are not subjected to a step of protease digestion prior to in vitro complexing to CD91 ligands.

To make a protein preparation of antigenic cells or virus particles, the lysing of antigenic cells or disruption of cell walls, cell membranes, or viral particle structure can be performed using standard protocols known in the art In various embodiments, the antigenic cells can be lysed, for example, by mechanical shearing, sonication, freezing and thawing, adjusting the osmolarity of the medium surrounding the cells, or a combination of techniques. In less preferred embodiments, the antigenic cells can be lysed by chemicals, such as detergents.

Once the cells are lysed, it is desirable to remove cellular debris, materials that are non-proteinaceous or materials that do not contain cytosolic, and/or membrane-derived proteins (including proteins in the membranes of organelles). Removal of these components can be accomplished by techniques such as low speed centrifugation or filtration. After removing cellular debris and intact cells, a high speed centrifugation step can be used to separate the cytosolic proteins which are in the supernatant, and the membrane-derived proteins which are collected in the pellet. Standard procedures commonly known in the art allows the further isolation of the membrane-derived proteins from the pellet. Standard techniques commonly known in the art can be used to extract viral proteins from viral particles. In certain embodiments, the methods used do not or are not designed to selectively remove or retain any one or more particular protein(s) from other proteins that are present in the antigenic cell, in the cytosol or in the membranes.

In other embodiments, optionally, the proteins from the antigenic cells can be separated by their general biochemical and/or biophysical properties, such as size, charge, or combinations thereof. Any techniques known in the art can be used to perform the separation. Selected fractions of the proteins/peptides that comprise at least 20, 50, 100, 500, 1,000, 5,000, 10,000, or 20,000 different proteins or that comprise at least 50%, 55%, 60%, 65%, 70%, 75%, 80%, 85%, 90%, 95% 97%, 98%, 99% of the different proteins present in the antigenic cells or a cellular fraction thereof, or virus particles, are used to form complexes to a CD91 ligand.

An exemplary, but not limiting, method that may be used to make a protein preparation comprising cytosolic proteins is as follows:
Cells, which may be tumor cells derived from a biopsy of the patient or tumor cells cultivated in vitro, or cell infected with a pathogenic agent, are suspended in 3 volumes of 1× Lysis buffer comprising 30 mM sodium bicarbonate pH 7.5, 1 mM PMSF, incubated on ice for 20 minutes and then the hypotonicauy-swollen cells are homogenized in a dounce homogenizer until >95% cells are lysed. As an alternative to shearing, cells can be sonicated, on ice, until >99% cells are lysed as determined by microscopic examination. When sonication is used, cells are suspended in a buffer such as phosphate buffered saline (PBS) which may comprises 1 mM PMSF, before sonication.
The lysate is centrifuged at 1,000×g for 10 minutes to remove intact cells, nuclei and other cellular debris. The resulting supernatant is recentrifuged at about 100,000×g for about one hour, and the supernatant recovered. The 100,000×g supernatant may be dialyzed for 36 hours at 4° C. (three times, 100 times volumes each time) against PBS or other suitable buffer, to provide the soluble cytosolic proteins of the present invention. If necessary, insoluble material in the preparation may be removed by filtration or low-speed centrifugation.

An exemplary, but not limiting, method that may be used to make a protein preparation comprising membrane-derived proteins is as follows:
Cells, which may be tumor cells derived from a biopsy of the patient or tumor cells cultivated in vitro, or cells infected with a pathogenic agent, are suspended in 3 volumes of 1× Lysis buffer comprising 30 mM sodium bicarbonate pH 7.5, 1 mM PMSF, incubated on ice for 20 minutes and then the hypotonically-swollen cells are homogenized in a dounce homogenizer until >95% cells are lysed. As an alternative to shearing, cells can be sonicated, on ice, until >99% cells are lysed as determined by microscopic examination. When sonication is used, cells are suspended in a buffer such as phosphate buffered saline (PBS) which may comprises 1 mM PMSF, before sonication.
The lysate is then centrifuged at 100,000×g for 10 minutes to collect the cell membranes. Membrane-derived proteins can be dislodged from the lipid bilayer and isolated from the 100,000 g pellet (where the membrane-derived proteins are located) by resuspending the pellet in 5 volumes of PBS containing 1% sodium deoxycholate (without Ca²⁺ and Mg²⁺) and incubated on ice for 1 h The resulting suspension is centrifuged for 30 min at 20,000 g and the resulting supernatant harvested and dialyzed against several changes of PBS (without Ca²⁺ and Mg²⁺) to remove the detergent. The resulting dialysate is centrifuged for 90 min at 100,000 g and the supernatant purified further. Then calcium and magnesium are both added to the supernatant to give final concentrations of 2 mM. If necessary, insoluble material in the preparation may be removed by filtration or low-speed centrifugation.

In a specific embodiment, the population of cytosolic and/or membrane-derived proteins obtained from antigenic cells can be complexed to a CD91 ligand directly without protease treatment or any further extraction or selection processes. Alternatively, the proteins can be subjected to protease treatment prior to complexing.

According to the invention, the cytosolic and membrane-derived proteins obtained from antigenic cells can be optionally digested to generate antigenic peptides. In one embodiment, either the cytosolic or the membrane-derived proteins are used in the digestion. In another embodiment, the cytosolic and membrane-derived proteins are combined in the digestion reaction to generate antigenic peptides. In preferred embodiments, the protein preparations that are used in the protease digestion have not been subjected to any method(s) of preparation that selectively remove or retain one or more particular protein(s) from the other proteins in the antigenic cells, or the cytosol or membranes of the antigenic cells.

Various proteases or proteolytic enzymes can be used in the invention to produce from a protein preparation of antigenic cells a population of peptides which comprises antigenic peptides. The enzymatic digestions can be performed either individually or in suitable combinations with any of the proteolytic enzymes that are well known in the art including, but not limited to, trypsin, Staphylococcal peptidase I (also known as protease V8), chymotrypsin, pepsin, cathepsin G, thermolysin, elastase, and papain. Trypsin is a highly specific serine proteinase that cleaves on the carboxyl-terminal side of lysines and arginines. Due to the limited number of cleavage sites, it is expected to leave many MHC-binding epitopes intact Staphylococcal peptidase L a serine proteinase, has specificity for cleavage after glutamic and aspartic acid residues. A digestion can be carried out with a single protease or a mixture of proteases. The proteases or proteolytic enzymes used are incubated under conditions suitable for the particular enzyme. Preferably, the enzyme is purified. Non-enzymatic methods, such as cyanogen bromide cleavage, can also be used for generating peptides. The protein preparation to be digested can be aliquoted into a plurality of reactions each using a different enzyme, and the resulting peptides may optionally be pooled together for use. It may not be necessary to completely digest the proteins in the enzymatic reactions. These reactions results in the generation of a diverse and different set of peptides for each protein that is present in the protein preparation. The production of different peptide sets allows for a greater probability of generating antigenic peptides that are capable of inducing an immune response to the antigens in the protein preparation when they are complexed to a CD91 ligand. In a preferred embodiment, the protein preparation to be digested is aliquoted into two separate reactions and two different proteolytic enzymes are used to produce two different sets of peptides of the proteins present in the protein preparation. Depending on the proteins, enzymes and reaction conditions, undigested proteins may remain in the reactions. In a preferred embodiment, trypsin and Staphylococcal peptidase I are used separately to digest the protein preparation

In another preferred embodiment, the proteolytic enzymes used in the invention exhibit similar activities as the proteolytic activities that are found in the proteasome. The proteasome is responsible for the extralysosomal, endocatalytic degradation of cytosolic and nuclear proteins which are mis-folded or damaged in a cell. The proteasome can degrade proteins completely to yield single amino acids, can generate optimal major histocompatibility complex class I (MHC I)-binding epitopes, and can generate longer peptide precursors which could potentially undergo further trimming elsewhere in the cell to yield cytotoxic T cell epitopes. Cleavage preferences of the proteasome is on the carboxyl (COOH)-side of basic, acidic, and hydrophobic amino acids. Three known proteolytic enzymatic activities that are present in the proteasome are chymotrypsin-like activity, trypsin-like activity, and peptidylglutamylpeptide-hydrolyzing activity (Uebel and Tampe, 1999, Curr. Opin. Immunol. 11:203-208). As such, enzymes having such activities and specificities can be used separately or in combination to digest the protein preparation. In a preferred embodiment, trypsin, chymotrypsin, and/or peptidylglutamylpeptide-hydrolase are used.

The resulting peptide digestions comprise antigenic peptides, non-antigenic peptides, and single amino acid residues. The reactions may also comprise undigested or incompletely digested antigenic proteins. The proteolytic enzymatic digestions of the invention are monitored in order to generate peptides that fall within a desirable range of lengths. In a preferred embodiment, the peptides generated are from about 7 to about 20 amino acid residues. Most antigenic peptides that are presented to T cells by MHC class I and class II fall within this range. In various embodiments, the population of peptides comprises peptides having a size range of 6 to 21, 8 to 19, 10 to 20, or at least 7, 8, 9, 10, 11, 12, 15, 20, 25, 30, 35, 40, 45, or 50, amino acid residues. In preferred embodiments, the antigenic peptides have 7, 8, 9, 10, 11, 12, 13, 14, 15, 16, 17, 18, 19, 20, or 35 amino acid residues. To monitor the progression of protein digestion, a test reaction can be performed where small aliquots of a protein digestion are taken out of the reaction and monitored for the progression of digestion through either tricine-polyacrylamide gel electrophoresis (“tricine-PAGE”), high performance liquid chromatography (“HPLC”), or mass spectrometry, or any other method known in the art to determine the size of peptides. Using such a test reaction, a determination can be made as to when peptide fragments of a particular size range will be generated at a particular enzymatic concentration. Other variables of the reaction that can be manipulated include the amount of protein in the reaction, the temperature, the duration of incubation, the presence of cofactors, etc.

Once the proper conditions are established for the generation of peptide fragments of a particular size range from a type of antigenic cell, the enzymatic reaction conditions can be duplicated to generate antigenic peptides which can be pooled. It is preferred that the enzymatic digestion is terminated bet;4,re the peptides are complexed to a CD91 ligand. In one embodiment of the invention, inhibitors cache used for terminating an enzymatic digestion Enzymatic inhibitors that can be used in the invention include, but are not limited to, PMSF, bestatin, amastatin, leupeptin, and cystatin, depending on which enzymes are used in the protein digestion. Inhibitors for most proteases are well known in the art. Alternatively, another method of terminating an enzymatic digestion is by physical removal of the enzyme from the reaction. This can be done by attaching the enzyme of choice to a solid phase, such as a resin or a material that can easily be removed from the reaction by well known methods such as centrifugation or filtration. The protein preparation is allowed to contact or flow across the solid phase for a period of time. Such immobilized enzymes can be purchased commercially or can be produced by procedures for immobilizing enzymes that are well known in the art.

At the end of the digestion reaction, the peptides can optionally be separated from low molecular weight materials, such as dipeptides, or single amino acid residues, in the preparation. For example, the peptides can be isolated by centrifugation through a membrane, such as the Centriprep-3. Optionally, the peptides can be separated by their general biochemical and/or biophysical properties, such as size, charge, or combinations thereof. Any techniques known in the art can be used to perform the separation resulting in digested protein preparation comprising at least 50, 100, 500, 1,000, 5,000, 10,000, 20,000, 50,000, or 100,000 different peptides.

In another embodiment of the invention, peptides that are endogenously present in antigenic cells can be used in the invention either alone or in combination with the peptides generated by the proteolytic digestion of the cytosolic and membrane-derived proteins. According to the invention, such peptides that are isolated directly from a protein preparation of antigenic cells can be complexed to CD91 ligands.

In specific embodiments, either the cytosolic or the membrane-derived proteins are used in the isolation process. In another specific embodiment, the cytosolic and membrane-derived proteins are combined in the isolation process. In preferred embodiments, the protein preparations that are used in the isolation have not been subjected to any method(s) of preparation that selectively remove or retain one or more particular protein(s) from the other proteins in the antigenic cells, or the cytosol or membranes of the antigenic cells. Preferably, the protein preparation comprises comprise at least 20, 50, 100, 500, 1,000, 5,000, 10,000, or 20,000 different proteins or that comprise at least 50%, 55%, 60%, 65%, 70%, 75%, 80%, 85%, 90%, 95% 97%/0, 98%, 99% of the different proteins present in the antigenic cells or a cellular faction thereof or virus particles.

In various embodiments, the method comprise treating the protein preparation to AT?, guanidium hydrochloride, and/or exposing the protein preparation to acidic conditions such that antigenic peptides in the protein preparation can be eluted. Many different acids can be used, including but not limited to, trifluoroacetic acid. Methods known in the art such as those described in Marston and Hartley, 1990, Meth. Enzymol. 182:264-276 for dissociating protein aggregates can be used.

In a particular embodiment, the isolation process comprises exposing a protein preparation of antigenic cells with AT?, for example, at room temperature for one hour, and/or treating a protein preparation of antigenic cells with trifluoroacetic acid (TFA), for example 0.1% TFA. The treatment preferably comprises sonicating the protein preparation in 0.1% TFA. In a most preferred embodiment, a protein preparation is first exposed to ATP, followed by sonication in 0.1% TFA. Various protease inhibitors can be used in the invention prior to cell lysis and the isolation process. For example, a mixture of 14 protease inhibitors can be used: phenylmethylsulfonyl fluoride (PMSF) 2 mM, ethylenediaminetetreacedic acid (EDTA) 1 mM, ethylene glycolbis(P-aminoethyl ether)N,N,N′,N′-tetraacetic acid (EGTA) 1 mM, (all obtained from Sigma, St. Louis, Mo.), and Antipain 20 mg/ml, Bestatin 5 mg/ml, Chemostatin 20 ptg/nil, E64 20 Jig/ml, Leupeptine 1 ttg/ml, Pepstatine 1 gg/ml, Pefabloc 40 Ag/ml, and Apoprotein 10 tkg/ml (all obtained from Boehringer Mannheim, Indianapolis, Ind.). The peptides resulting from the protein preparation comprise antigenic peptides and non-antigenic peptides of a variety of sizes ranging from at least 7, 8, 9, 10, 11, 12, 15, 20, 25, 30, 35, 40, 45, or 50, amino acid residues. In preferred embodiments, the peptides have 7, 8, 9, 10, 11, 12, 13, 14, 15, 16, 17, 18, 19, 20, or 35 amino acid residues. At the end of the process, the peptides are preferably recovered by separating from the proteins in the preparation prior to complexing with a CD91 ligand. For example, the peptides can be recovered by centrifugation through a membrane, such as the Centriprep-3, by drying under vacuum, or by reverse phase chromatography, e.g., fractionation in a BioCad20 microanalytiocal HPLC Poros RH2 column (Perseptive Biosystems, Cambridge, Mass.), equilibrated with 0.1% TFA in water and elution by acetonitrile. Accordingly, antigenic peptides that are endogenously present in antigenic cells and that are isolated directly from a protein preparation can be complexed to a CD91 ligand. Alternatively, a mixed population of peptides comprising peptides that are endogenously present in antigenic cells and peptides from digested cytosolic and membrane-derived proteins, can be complexed to a CD91 ligand.

4.1.2 Synthetic Production of Antigenic Proteins

Once the nucleotide sequence or amino acid sequence of an antigenic protein has been determined or obtained, the peptide can be produced, either by recombinant techniques or by synthetic methods. The antigenic peptide may be synthesized using conventional peptide synthesis or any of a number of other protocols well known in the art. For example, a peptide can be synthesized by use of a peptide synthesizer. Either the entire protein can be synthesized, or an antigenic determinant thereof, preferably the portion of the protein that contains the mutant or variant amino acid(s).

Peptides may be synthesized by solid-phase peptide synthesis using procedures similar to those described by Merrifield, 1963, J. Am. Chem. Soc. 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art (see Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

In addition, analogs and derivatives antigenic proteins can be chemically synthesized. Furthermore, if desired, nonclassical amino acids or chemical amino acid analogs can be introduced as a substitution or addition into the sequence of the antigenic proteins. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutync acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Purification of the resulting peptides is accomplished using conventional procedures, such as preparative “PLC using gel permeation, partition and/or ion exchange chromatography. The techniques, choice of appropriate matrices and buffers are well known in the art (Atherton et al., 1989, Solid Phase Peptide Synthesis: A Practical Approach, IRL Press). Preferably, the synthetic peptide prepared is 50%, 55%, 60%, 65%, 70%, 75%, 80%, 85%, 90%, 95% ,96%, 97%, 98%, 99% or 100% pure.

4.13 Recombinant Production of Antigenic Proteins

As an alternative to synthetic production, antigenic protein can produced by recombinant means. Once the nucleotide sequence encoding an antigenic protein has been identified, the nucleotide sequence, or a fragment thereof can be obtained and cloned into an expression vector for recombinant expression. The expression vector can then be introduced into a host cell for propagation of the antigen. Methods for recombinant production of antigenic proteins are described in detail herein.

Any recombinant antigenic protein useful in the complexes of the invention is preferably 50%, 55%, 60%, 65%, 70%, 75%, 80%, 85%, 90%, 95% ,96%, 97%, 98%, 99% or 100% pure. The DNA may be obtained by DNA amplification or molecular cloning directly from a tissue, cell culture, or cloned DNA (e.g., a DNA “library”) using standard molecular biology techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Current Protocols in Molecular Biology, Ausubel et al. (eds.), Greene Publishing Associates and Wiley lnterscience, New York, each of which is incorporated herein by reference in its entirety). Clones derived from genomic DNA may contain regulatory and intron DNA regions in addition to coding regions; clones derived from cDNA will contain only exon sequences. Whatever the source, the antigen gene should be cloned into a suitable vector for propagation of the gene.

In a preferred embodiment, DNA can be amplified from genomic or cDNA by polymerase chain reaction (PCR) amplification using primers designed from the known sequence of a related or homologous antigen. PCR is used to amplify the desired sequence in DNA clone or a genomic or cDNA library, prior to selection. PCR can be carried out, e.g., by use of a thermal cycler and Taq polymerase (Gene Amp®). The polymerase chain reaction (PCR) is commonly used for obtaining genes or gene fragments of interest. For example, a nucleotide sequence encoding an antigenic protein of any desired length can be generated using PCR primers that flank the nucleotide sequence encoding the peptide-binding domain. Alternatively, an antigenic protein gene sequence can be cleaved at appropriate sites with restriction endonuclease(s) if such sites are available, releasing a fragment of DNA encoding the antigenic protein gene, or an antigenic derivative thereof. If convenient restriction sites are not available, they may be created in the appropriate positions by site-directed mutagenesis and/or DNA amplification methods known in the art (see, for example, Shankrppa et al., 1992, PCR Method Appl. 1:277-278). The DNA fragment that encodes the antigenic protein is then isolated, and ligated into an appropriate expression vector, care being taken to ensure that the proper translation reading frame is maintained.

In an alternative embodiment, for the molecular cloning of an antigenic protein gene from genomic DNA, DNA fragments are generated to form a genomic library. Since some of the sequences encoding related protein antigens are available and can be purified and labeled, the cloned DNA fragments in the genomic DNA library may be screened by nucleic acid hybridization to a labeled probe (fenton and Davis, 1977, Science 196:180; Grunstein and Hogness, 1975, Proc. Natl Acad. Sci. U.S.A. 72:3961). Those DNA fragments with substantial homology to the probe will hybridize. It is also possible to identify an appropriate fragment by restriction enzyme digestion(s) and comparison of fragment sizes with those expected according to a known restriction map.

Alternatives to isolating the antigenic protein genomic DNA include, but are not limited to, chemically synthesizing the gene sequence itself from a known sequence or synthesizing a cDNA to the mRNA which encodes the antigenic protein. For example, RNA for cDNA cloning of the antigenic protein gene can be isolated from cells which express the antigenic protein. A cDNA library may be generated by methods known in the art and screened by methods, such as those disclosed for screening a genomic DNA library. If an antibody to the antigenic protein is available,the antigenic protein may be identified by binding of a labeled antibody to the antigenic protein synthesizing clones.

Other specific embodiments for the cloning of a nucleotide sequence encoding an antigenic protein, are presented as examples but not by way of limitation, as follows:

In a specific embodiment, nucleotide sequences encoding an antigenic protein can be identified and obtained by hybridization with a probe comprising a nucleotide sequence encoding the antigenic protein under conditions of low to medium stringency. By way of example and not limitation, procedures using such conditions of low stringency are as follows (see also Shilo and Weinberg, 1981, Proc. Natl. Acad. Sci. U.S.A. 78:6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem. 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques 8:404-407), etc. Modifications can be confirmed by double stranded dideoxynucleotide DNA sequencing.

4.1.3.1 Host-Vector Systems

Nucleotide sequences encoding an antigenic protein can be inserted into the expression vector for propagation and expression in recombinant cells. An expression construct, as used herein, refers to a nucleotide sequence encoding an antigenic protein operably associated with one or more regulatory regions which allows expression of the antigenic protein in an appropriate host cell. “Operably-associated” refers to an association in which the regulatory regions and the antigenic protein sequence to be expressed are joined and positioned in such a way as to permit transcription, and ultimately, translation of the protein sequence. A variety of expression vectors may be used for the expression of proteins, including, but not limited to, plasmids, cosmids, phage, phagemids, or modified viruses. Examples include bacteriophages such as lambda derivatives, or plasmids such as pBR322 or pUC plasmid derivatives or the Bluescript vector (Stratagene). Typically, such expression vectors comprise a functional origin of replication for propagation of the vector in an appropriate host cell, one or more restriction endonuclease sites for insertion of the antigemc protein gene sequence, and one or more selection markers.

Vectors based on E. coli are the most popular and versatile systems for high level expression of foreign proteins (Makrides, 1996, Microbiol. Rev. 60:512-538). Non-limiting examples of prokaryotic expression vectors may include the λgt vector series such as λgt11 (Huynh et al., 1984 in “DNA Cloning Techniques”, Vol. I: A Practical Approach (D). Glover, ed.), pp. 49-78, IRL Press, Oxford), and the pET vector series (Studier et al., 1990, Methods Enzymol., 185:60-89). Non-limiting examples of regulatory regions that can be used for expression in E. coli may include but not limited to lac, trp, lpp, phoA, recA, tac, λP_{L}, and phage T3 and T7 promoters (Malcrides, 1996, supra).

However, a potential drawback of a prokaryotic host-vector system is the inability to perform many of the post-translational processing events of mammalian cells. Thus, a eukaryotic host-vector system is preferred, a mammalian host-vector system is more preferred, and a human host-vector system is the most preferred. The regulatory regions necessary for transcription of an antigenic protein or polypeptide can be provided by the expression vector. A translation initiation codon (ATG) may also be provided to express a nucleotide sequence encoding an antigenic protein that lacks an initiation codon. In a compatible host-construct system, cellular proteins required for transcription, such as RNA polymerase and transcription factors, will bind to the regulatory regions on the expression construct to effect transcription of the antigenic protein sequence in the host organism. The precise nature of the regulatory regions needed for gene expression may vary from host cell to host cell. Generally, a promoter is required which is capable of binding RNA polymerase to initiate the transcription of an operably-associated nucleic acid sequence. Such regulatory regions may include those 5′-non-coding sequences involved with initiation of transcription and translation, such as a TATA box, cap site, a CAAT box, transcription factor binding sites, enhancer elements, and the like. The non-coding region 3′ to the coding sequence may contain transcriptional termination regulatory sequences, such as terminators and polyadenylation sites.

Both constitutive and inducible regulatory regions may be used for expression of the antigenic protein. It may be desirable to use inducible promoters when the conditions optimal for growth of the recombinant cells and the conditions for high level expression of the antigenic protein are different. Examples of useful regulatory regions are provided in the next section below.

For expression of antigenic proteins in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LIR), the β-interferon gene, and the Hsp70 gene (Williams et al., 1989, Cancer Res. 49:2735-42 ; Taylor et al., 1990, Mol. Cell. Biol. 10:165-75).

The following animal regulatory regions, which exhibit tissue specificity and have been utilized in transgenic animals, can also be used in cells of a particular tissue type of interest: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells Ceder et al., 1986, Cell 45:485-495), albumin gene control region which is active in liver (Pinkert et al., 1987, Genes Dev. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrrpsin gene control region which is active in the liver (Kelsey et al., 1987, Genes Dev. 1:161-171), beta-globin gene control region which is active in myeloid cells (Mogram et al., 1985, Nature 315:338-340; Kollias et al., 1986, Cell 46: 89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

The efficiency of expression of the antigenic protein in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. Biotechnol. 1:6-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding an antigenic protein. For long term, high yield production of antigenic proteins, stable expression in mammalian cells is preferred. A number of selection systems may be used for mammalian cells, including, but not limited, to the Herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. U.S.A. 48:2026), and adenine phosphoribosyltransferase (Lowy et al., 1980, Cell 22:817) genes can be employed in tk, hgprf or aprf cells, respectively. Also, antimetabolite resistance can be used as the basis of selection for dihydrofolate reductase (dhfr), which confers resistance to methotrexate (Wigler et al., 1980, Natl. Acad. Sci. U.S.A. 77:3567; O'Hare et al., 1981, Proc. Natl. Acad. Sci. U.S.A. 78:1527); gpt, which confers resistance to mycophenolic acid (Mulligan and Berg, 1981, Proc. Natl. Acad. Sci. U.S.A. 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

In order to insert the DNA sequence of the antigenic protein into the cloning site of a vector, DNA sequences with regulatory functions, such as promoters, must be attached to DNA sequences encoding the antigenic protein. To do this, linkers or adapters providing the appropriate compatible restriction sites may be ligated to the ends of cDNA or synthetic DNA encoding an antigenic protein, by techniques well known in the art (Wu et al., 1987, Methods Enzymol. 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

The expression construct comprising an antigenic protein sequence operably associated with regulatory regions can be directly introduced into appropriate host cells for expression and production of antigenic protein-CD91 ligand complexes without further cloning (see, for example, U.S. Pat. No. 5,580,859). The expression constructs may also contain DNA sequences that facilitate integration of the antigenic protein nucleotide sequence into the genome of the host cell, e.g., via homologous recombination. In this instance, it is not necessary to employ an expression vector comprising a replication origin suitable for appropriate host cells in order to propagate and express the antigenic protein in the host cells.

Expression constructs containing cloned nucleotide sequence encoding antigenic polypeptides can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215: 166-168), electroporation (Wolff et al., 1987, Proc. Natl. Acad. Sci. 84:3344), and microinjection (Cappechi, 1980, Cell 22:479-488). Co-expression of an antigenic peptide and a CD91 ligand in the same host cell can be achieved by essentially the same methods.

For long term, high yield production of properly processed antigenic proteins or antigenic protein-CD91 ligand complexes, stable expression in mammalian cells is preferred. Cell lines that stably express antigenic proteins or antigenic protein-CD91 ligand complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while antigenic protein is expressed continuously.

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley lnterscience, which is incorporated herein by reference; and the catalogs of commercial suppliers such as Clontech Laboratories, Stratagene Inc., and Invitrogen, Inc.

Alternatively, a number of viral-based expression systems may also be utilized with mammalian cells to produce antigenic proteins. Vectors using DNA virus backbones have been derived from simian virus 40 (SV40) (Hamer et al., 1979, Cell 17:725), adenovirus (Van Doren et al., 1984, Mol. Cell Biol. 4:1653), adeno-associated virus (McLaughlin et al., 1988, J. Virol. 62:1963), and bovine papillomas virus (Zinn et al., 1982, Proc. Natl. Acad. Sci. 79:4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts (see, e.g., Logan and Shenk, 1984, Proc. Natl. Acad. Sci. U.S.A. 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copieslcell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct is transfected into cultured mammalian cells, for example, by the techniques of calcium phosphate coprecipitation or electroporation. For those host cells that do not manifest a transformed phenotype, selection of transformants is achieved by use of a dominant selectable marker, such as histidinol and G418 resistance. For example, BPV vectors such as pBCMGSNeo and pBCMGHis may be used to express antigenic peptide sequences (Karasuyama et al., Eur. J. Immunol. 18:97-104; Ohe et al., Human Gene Therapy 6:325-33) which may then be transfected into a diverse range of cell types for expression of the antigenic protein.

Alternatively, the vaccinia 7.5K promoter may be used (see, e.g. Mackett et al., 1982, Proc. Natl. Acad. Sci. U.S.A. 79:7415-7419; Mackett et al., 1984, J. Virol. 49:857-864; Panicali et al., 1982, Proc. Natl. Acad. Sci. U.S.A. 79:49274931) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., BBO-pCD (Spickofky et al., 1990, DNA Prot. Eng. Tech. 2:14-18), pDR2 and λDR2 (available from Clontech Laboratories).

Antigenic proteins may also be made with a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with nucleic acid sequences encoding the antigenic protein, while the missing viral functions can be supplied in trans. The host range for infection by a retroviral vector can also be manipulated by the choice of envelope used for vector packaging.

For example, a retroviral vector can comprise a 5′ long terminal repeat (LfR), a 3′ LTR, a packaging signal, a bacterial origin of replication, and a selectable marker. The antigenic peptide DNA is inserted into a position between the 5′ LIR and 3′ LTR, such that transcription from the 5′ LIR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38:91-135; Morgenstern et al., 1990, Nucleic Acid Res. 18: 3587-3596; Choulika et al., 1996, J. Virol 70:1792-1798; Boesen et al., 1994, Biotherapy 6: 291-302; Salmons and Gunzberg, 1993, Human Gene Therapy 4:129-141; and Grossman and Wilson, 1993, Curr. Opin. in Genetics and Devel. 3:110-114).

Other useful eukaryotic host-vector system may include yeast and insect systems. In yeast, a number of vectors containing constitutive or inducible promoters may be used with Saccharomyces cerevisiae (baker's yeast), Schizosaccharomyces pombe (fission yeast), Pichia pastoris, and Haitsetiula polymorpha (methylotropic yeasts). For a review see, Current Protocols in Molecular Biology, Vol. 2, 1988, Ed. S et al., Greene Publish Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kijmnel, Acad. Press, N.Y., Vol. 152, pp. 673-684; and The Molecular Biology of the Yeast Saccharomyces, 1982, Eds. Strathem et al., Cold Spring Harbor Press, Vols. I and II.

In an insect system a baculovirus, Atuograplia californica nuclear polyhidrosis virus (AcNPV), can be used as a vector to express an antigenic peptide in Spodoptera frugiperda cells. The antigenic protein DNA may be cloned into non-essential regions (for example the polyhedrin gene) of the virus and placed under control of an AcNPV promoter (for example the polyhedrin promoter). These recombinant viruses are then used to infect host cells in which the inserted DNA is expressed (see, e.g., Smith et al., 1983, J. Virol. 46:584; Smith, U.S. Pat. No. 4,215,051).

The expression vector must be used with a compatible host cell which may be derived from a prokaryotic or an eukaryotic organism, including, but not limited to bacteria, yeasts, insects, mammals, and humans. Any cell type that is compatible with the expression vector may be used, including those that have been cultured in vitro or genetically engineered. Host cells may be obtained from normal or affected subjects, including healthy humans, patients with cancer or an infectious disease, private laboratory deposits, public culture collections such as the American Type Culture Collection, or from commercial suppliers.

Preferred mammalian host cells include but are not limited to those derived from humans, monkeys and rodents, (see, for example, Kriegler M. in “Gene Transfer and Expression: A Laboratory Manual”, New York, Freeman & Co. 1990), such as monkey kidney cell line transformed by SV40 (COS-7, ATCC CRL 1651), human embryonic kidney line (293, 293-EBNA), or 293 cells subeloned for growth in suspension culture (Graham et al., 1977, J. Gen. Virol. 36:59), baby hamster kidney cells (BHK, ATCC CCL 10), chinese hamster ovary-cells-DHFR (CHO, Urlaub and Chasin, 1980, Proc. Natl. Acad. Sci. 77:4216), mouse sertoli cells (Mather, 1980, Biol. Reprod. 23:243-251), mouse fibroblast cells (NIH-3T3), monkey kidney cells (CVI ATCC CCL 70), african green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442); human lung cells (W138, ATCC CCL 75), human liver cells (Hep G2, HB 8065), and mouse mammary tumor cells (MMF 060562, ATCC CCL51). Exemplary cancer cell types used for demonstrating the utility of recombinant cells as a cancer vaccine are provided as follows: mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC#2 and SCLC#7.

The recombinant cells may be cultured under standard conditions of temperature, incubation time, optical density, and media composition. Alternatively, a cells may be cultured under conditions emulating the nutritional and physiological requirements of a cell in which the antigenic protein is endogenously expressed The antigenic protein, or an antigenic portion thereof, can be purified by any methods appropriate for the protein, and then used to form complexes with CD91 ligands as described in section 4.4, below.

4.1.3.2 Purification Methods for Recombinant Antigenic Proteins

Generally, the recombinant antigenic proteins of the invention can be recovered and purified from recombinant cell cultures by known methods, including ammonium sulfate precipitation, acid extraction, anion or cation exchange chromatography, phosphocellulose chromatography, immunoaffinty chromatography, hydroxyapatite chromatography, and lectin chromatography.

In one embodiment, the invention provides methods for purification of recombinant antigenic proteins by affinity purification. The principle of affinity chromatography well known in the art. One approach is based on specific molecular interactions between an affinity label present on the antigenic protein and its binding partner. A second approach, immunoaffinity chromatography, relies on the immunospecific binding of an antibody to an epitope present on the tag.

Described below are several methods based on specific molecular interactions of a tag and its binding partner. Protein A affinity chromatography, a method that is generally applicable to purifying recombinant antigenic proteins that are fused to the constant regions of immunoglobulin, is a well known technique in the art. Staphylococcus protein A is a 42 kD polypeptide that binds specifically to a region located between the second and third constant regions of heavy chain immunoglobulins. Because of the Fe domains of different classes, subclasses and species of immunoglobulins, affinity of protein A for human Fe regions is strong, but may vary with other species. Subclasses that are less preferred include human IgG-3, and most rat subclasses. For certain subclasses, protein G (of Streptococci) may be used in place of protein A in the purification. Protein-A sepharose (Pharmacia or Biorad) is a commonly used solid phase for affinity purification of antibodies, and can be used essentially in the same manner for the purification of an antigenic protein of interest fused to an immunoglobulin Fc fragment. Antigenic protein of interest present in cell lysate or, if secreted by the cell, in the supernatant, binds specifically to protein A on the solid phase, while the contaminants are washed away. Bound antigenic protein of interest can be eluted by various buffer systems known in the art, including a succession of citrate, acetate and glycine-HCl buffers which gradually lowers the pH. This method is less preferred if the recombinant cells also produce antibodies which will be copurified with the antigenic protein. See, for example, Langone, 1982, J. Immunol. Meth. 51:3; Wilchek et al., 1982, Biochem. Intl. 4: 629; Sjobring et al., 1991, J. Biol. Chem. 26:399; page 617-618, in Antibodies A Laboratory Manual, edited by Harlow and Lane, Cold Spring Harbor laboratory, 1988.

Alternatively, a polyhistidine tag may be used, in which case, the antigenic protein can be purified by metal chelate chromatography. The polyhistidine tag, usually a sequence of six histidines, has a high affinity for divalent metal ions, such as nickel ions (Ni²⁺), which can be immobilized on a solid phase, such as nitrilotriacetic acid matrices. Polyhistidine has a well characterized affinity for Ni²⁺-NTA-agarose, and can be eluted with either of two mild treatments: imidazole (0.1-0.2 M) will effectively compete with the resin for binding sites; or lowering the pH just below 6.0 will protonate the histidine side-chains and disrupt the binding. The purification method comprises loading the cell culture supernatant onto the Ni²⁺-NTA-agarose column, washing the contaminants through, and eluting the antigenic protein of interest with imidazole or weak acid. Ni²⁺-NTA-agarose can be obtained from commercial suppliers such as Sigma (St. Louis) and Qiagen. Antibodies that recognize the polyhistidine tag are also available which can be used to detect and quantify the antigenic protein.

Another exemplary affinity label that can be used is the glutathione-S-transferase (GST) sequence, originally cloned from the helminth, Schistosonia japonicum. In general, an antigenic protein-GST fusion expressed in a prokaryotic host cell, such as E. coli, can be purified from the cell culture supernatant by absorption with glutathione agarose beads, followed by elution in the presence of free reduced glutathione at neutral pH. Denaturing conditions are not required at any stage during purification, and therefore, it may be desirable co-purification of CD91 ligands and antigenic proteins, for use in the loading of immobized CD91 ligands with antigenic proteins. Moreover, since GST is known to form dimers under certain conditions, dimeric antigenic proteins may be obtained. See, Smith, 1993, Methods Mol. Cell Bio. 4:220-229.

Another useful affinity label that can be used is the maltose binding protein (MBP) of E. coli, which is encoded by the male gene. The secreted protein-MBP present in the cell supernatant binds to amylose resin while contaminants are washed away. The bound antigenic protein-MBP is eluted from the amylose resin by maltose. See, e.g., Guan et al., 1987, Gene 67:21-30.

The second approach for purifying antigenic proteins is applicable to affinity labels that contain an epitope for which polyclonal or monoclonal antibodies are available. Various methods known in the art for purification of protein by immunospecific binding, such as immunoaffinity chromatography, and immunoprecipitation, can be used. See, e.g., Chapter 13 in Antibodies A Laboratory Manual, edited by Harlow and Lane, Cold Spring Harbor laboratory, 1988; and Chapter 8, Sections I and II, in Current Protocols in Immunology, ed by Coligan et al., John Wiley, 1991; the disclosure of which are both incorporated by reference herein.

4.2 Sources of CD91 Ligands

CD91 ligands suitable for the complexes and fusion proteins of the invention can be isolated from the cells and/or serum of a subject by various methods described in the art (see, e.g., Bensadoun et al., Methods in Molecular Biology, Vol. 109, Humana Press Inc., Totowa, N.J., (Doolittle and Reue eds.), pages 145-50).

Amino acid sequences and nucleotide sequences of many CD91 ligands are generally available in sequence databases, such as GenBank. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics.

For example, the following is a list of GenBank accession numbers for some CD91 ligands: ApoE (GenBank Accession Number: LPHUE), Lipoprotein lipase (GenBank Accession Number. LIHUL), Hepatic lipase (GenBank Accession Number: AAC34206), Factor IXa (GenBank Accession Number: KFHU), Factor VIIIa (GenBank Accession Number: P00451, GenBank Accession Number: EZHU), Factor VIIa/TFPI (GenBank Accession Number: KFHU7, GenBank Accession Number: P10646), MMP-13 (GenBank Accession Number: P45452), MMP-9 (GenBank Accession Number. CAC07541), Pregnancy Zone Protein (GenBank Accession Number: S29738), PAI-1, Antithrombin III (GenBank Accession Number: AAB40025), Tissue factor pathway inhibitor (TFPI)(GenBank Accession Number: KFHHU7), Heparin cofactor II (GenBank Accession Number: NP_{—}000176), Thrombospondin-1 (GenBank Accession Number: NP_{—}003237), Thrombospondin-2 (GenBank Accession Number: NP_{—}003238), Lactoferrin (GenBank Accession Number: AAN84790) and HIV-Tat protein (GenBank Accession Number: BAA12992, GenBank Accession Number: BAA13000) Once the nucleotide sequence of a CD91 ligand has been identified, CD91 ligands can be produced by any method known in the art, such as the recombinant methods described above (Section 4.1.3) for the production of antigenic proteins. CD91 ligand-encoding cDNA or genomic DNA may be obtained from any species, for example, by PCR amplification. Oligonucleotide primers representing known nucleic acid sequences of related CD91 ligands can be used as PCR primers. One can choose to synthesize several different degenerate primers for use in the PCR reactions. It is also possible to vary the stringency of hybridization conditions used in priming the PCR reactions, to allow for greater or lesser degrees of nucleotide sequence similarity between the known CD91 ligand nucleotide sequence and the nucleic acid homolog being isolated. For cross species hybridization, low stringency conditions are preferred. For same species hybridization, moderately stringent conditions are preferred. After successful amplification, the sequence encoding a CD91 ligand may be cloned and sequenced. If the size of the coding region of the CD91 ligand gene being amplified is too large to be amplified in a single PCR, several PCR covering the entire gene, preferably with overlapping regions, may be carried out, and the products of the PCR ligated together to form the entire coding sequence. Alternatively, if a segment of a CD91 ligand gene is amplified, that segment maybe cloned, and utilized as a probe to isolate a complete cDNA or genomic clone. The CD91 ligand gene may then be cloned into an appropriate expression vector, and be produced, propogated, isolated and purified according to methods for recombinant production of proteins, such as those described for recombinant production of antigenic proteins described in Section 4.1.3, above.

An alternative to producing CD91 ligands by recombinant techniques is peptide synthesis. For example, a CD91 ligand, or a fragment thereof, can be synthesized using methods known in the art, such as those described for the synthesis of antigenic proteins, in Section 4.1.2, above. In this case, conventional peptide synthesis may be used, or other synthetic protocols well known in the art.

Purification of the resulting CD91 ligand, or fragment thereof may then be accomplished using conventional procedures, such as preparative HPLC using gel permeation, partition and/or ion exchange chromatography. The choice of appropriate matrices and buffers are well known in the art.

4.3 Complexes and Fusion Proteins Comprising a CD91 Ligand Fragment and an Antigenic Molecule

The invention contemplates complexes and fusion proteins comprising a CD91 ligand fragment and an antigenic molecule. Preferably the CD91 ligand fragment contains a receptor binding domain Receptor binding domains have been identified for various CD91 ligands (see, e.g., Swertfeger and Hui, 2001, J. Biol. Chem. 276:25043 and Weisgraber et al., 1983, J. Biol. Chem. 258:12348 (receptor binding domain for Apo E at amino acid residues 141-155); Williams et al., 1994, J. Biol. Chem 269:8653 and Krapp et al., 1995, 3. Lipid Res. 36:2362-73 (receptor binding domain for lipoprotein lipase at amino acid residues 313-448); Krapp et al., 1996, J. Lipid Res. 37:926-36 (receptor binding domain lipoprotein lipase at amino acid residues 378-448); Nykaer et al., 1994, J. Biol. Chem. 269:31747-55 (receptor binding domain for lipoprotein lipase at amino acid residue 378-423); Krapp et al., 1996, J. Lipid Res. 37:926-36 (sequences within the carboxyl-terminal 73 amino acids are important for surface binding of human hepatic lipase, and four basic amino acids [K⁴⁷², R⁴⁷³, R⁴⁷⁴] located at the carboxyl terminal region may play a critical role); Bassel-Duby et al., 1992, J. Biol. Chem. 267:9668, Grobmyer et al., 1993, J. Biol. Chem. 268: 13291, Nykjaer et al., 1994, J. Biol. Chem. 269:25668, Larsen et al, 1989, Blood 73:1842, Camani et al., 1994, J. Biol. Chem 269:5770 (receptor binding domain for tPA at amino acid residues 4-50 (fibronectin fingerlike domain), 51-87 (EGF domain), 4-87 (finger+EGF domain)); Nykjaer et al., 1994, J. Biol. Chem. 269:25668 (receptor binding domain for uPA α chain at amino acid residues 50-131 (kringle domain) and β chain at amino acid residues 159-411 (serine protease domain); Warshawsky et al., 1995, Biochem. 34:3403, Warshawsky et al, 1993, J. Biol. Chem. 268:22046 and Warshawsky et al., 1994, J. Biol. Chem. 269:3325 (receptor binding domain for RAP at amino acid residues 1-114, 12-107, 200-319 and 115-319); Liu et al., 2000, Nat. Med. 6(12):1380-87 (receptor binding domain for HIV TAT, core domain at amino acid residues 3748 and basic domain at amino acid residues 48-57); Mikhailenko et al., 1997, J. Biol. Chem. 272:6784-91 and Chen et al., 1996, Biochem. J., 318:959-963 (receptor binding domain for thrombospondin-1, N-terminal heparin binding domain (mouse) at amino acid residues 1-214); Neels et al., 2000, Blood 96:3459-65 (CD91 does not bind factor IX zymogen-it only binds the active species; factor IXa binds to CD91 at a site outside of the active site); Sarafanov et al., 2001, J. Biol. Chem 276:11970-79 (receptor binding domain for factor VIIIa, A2 domain at amino acid residues 373-740); Saenko et al., 1999, J. Biol. Chem 274:37685-92 (receptor binding domain for factor VIIIa, A2 domain at amino acid residues 484˜509); Lenting et al, 1999, J. Biol. Chef 274:23734-39 (receptor binding domain for factor Villa, light chain-C2 domain at amino acid residues 2173- 2332); Iakiiaev et al., 1999, J. Biol. Chem 274:36995-37003 and Hamik et al., 1999, J. Biol. Chen 274:4962-69 (receptor binding domain for TFPI at C terminal domain); Steffansson et al., 1998, J. Biol. Chem. 273:6358-66, Hornet al., 1996, Fibrinolysis 10:21, Rodenburg et al., 1998, Biochem. J. 329:55-63 and Hom et al., 1998, Thromb. Haemost. 80:822-8 (receptor binding domain for PAI-I, heparin binding domain (basic residues); high affinity site generated in PAI-1 when in complex with a proteinase); Jensen et al., 1996, Biochim. Biophys. Acta. 1293:254-8 and Arbelaez et al., 1997, Protein Exp. Purif. 10:301-8 (receptor binding domain for pregnancy zone protein (?ZP) at C-terminal fragment); Nielsen et al., 1996, J. Biol. Chem. 27 1:12909-12, VanLeuven et al., 1986, J. Biol. Chem. 261:11369-73 and Sottrup-Jensen et al., 1986, FEBS Lett 205:20-24 (receptor binding domain for α2M, 20 kDa C-terminal stretch at amino acid residues 1314-1451 ( human α2M)); Meilinger et al., 1999, J. Biol. Chem. 274:38091-96 (receptor binding domain for complement C3 present in activated C3 (thioester cleaved forms)); and Kinoshita et al., 2001, J. Neurosci. 21 :835461 and Kounnas et al., 1995, Cell 82:331-40 (receptor binding domain for APP on extracellular and intracellular domains; isoforms containing a Kunitz proteinase inhibitor (KPI) domain). The preceding references are incorporated by reference in their entirety.

Many assays are known in the art for identifying receptor binding domains of molecules of interest. Any such assay can be used to identify suitable CD91 ligand fragments for use in the invention. Once the binding domain is identified, a fragment of the CD91 ligand can be made or constructed using any method known in the art, including, by way of example and not limitation, proteolytic cleavage, and recombinant or synthetic techniques.

One such method for identifying a binding domain of a CD91 ligand comprises constructing mutants of the CD91 ligand (for example, the mutants may contain a deletion, truncation or point mutation). The mutants are then assayed for their ability to interact with a receptor which is known to bind to the CD91 ligand. In this manner, the binding domain of the ligand can be identified.

Another such method for identifying a binding domain of a CD91 ligand comprises assaying for the ability of a particular fragment or epitope of a CD91 ligand to modulate receptor-ligand binding events.

Another example of a method for identifying a binding domain of a CD91 ligand comprises the use of an antibody that binds to a particular epitope on the CD91 ligand. The CD91 ligand is first exposed to the antibody, and then the CD91 ligand is assayed for its ability to bind a receptor of interest.

Any method known in the art for detecting receptor binding may be used (see, e.g., Stanford and Horton, 2002, Receptors: Structure and Function, A Practical Approach, Oxford University Press and Current Protocols in Molecular Biology, Ausubel et al. (eds.), Greene Publishing Associates and Wiley Interscience, New York, which are both incorporated by reference herein).

Specifically, cell-free assays known in the art may be used to detect receptor-ligand binding such as those described by Sjolander and Urbaniczky, 1991. Anal. Chem. 63:2338-2345 and Szabo et al., 1995, Curr. Opin. Struct Biol. 5:699-705, which are incorporated by reference herein. Other cell free assays that may be used include assays in which an immobilized protein is tested for its ability to bind to a putative cognate protein (e.g., ELISA and affinity matrices), and two hybrid or three hybrid assays (see, e.g. U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabachi et al., 1993, Oncogene 8:1693-1696; and PCT WO94/10300; the preceding disclosures are hereby incorporated by reference).

In addition, any cell based assay known in the art may be used to detect receptor-ligand interactions. In such an assay, a receptor protein or biologically active portion thereof is contacted with either 1) mutant ligand molecules, 2) fragments or epitopes of ligand, or 3) ligand molecules in the presence of an antibody, or fragments or epitopes of ligand. The receptor-ligand binding interaction is then monitored, for example, by cellular phenotype or a receptor mediated cellular response (e.g., uptake of ligand or modulation of signal transduction).

4.4 In Vitro Complexing

In an alternative embodiment, complexes of CD91 ligands with antigenic molecules are produced in vitro. Immunogenic CD91 ligand-antigenic molecule complexes can be generated in vitro by covalent or non-covalent coupling of a CD91 ligand with an antigenic molecule. As described in Section 4.1 above, antigenic molecules may be isolated from various sources, chemically synthesized, or produced recombinantly. CD91 ligands may also be prepared by a variety of methods, as described in Section 4.2, above. After isolation of CD91 ligands and antigenic molecules, complexes are produced in vitro. Procedures for forming such CD91 ligand-antigenic molecule complexes and preferred, exemplary protocols for covalently and noncovalently forming such complexes are provided herein. Such methods can be readily adapted for medium or large scale production of the immunotherapeutic or prophylactic vaccines of the invention.

4.4.1 Formation of Non-Covalent CD91 Ligand-Antigenic Molecule Complexes

Any method known in the art can be used for preparing non-covalent complexes of the invention The formation of non-covalent immunogenic complexes of CD91 ligands and antigenic molecules can be achieved under conditions that favor complex formation

The following example describes a method for preparing non-covalent immunogenic complexes of CD91 ligands and antigenic molecules:
CD91 ligands are prepared, as described in Section 4.2, above. The antigenic molecules and the CD91 ligand are mixed together. The mixture is then incubated in a suitable buffer. The preparations are then centrifuged through filter to remove any unbound peptide. If the CD91 ligand is bound to a solid phase, the CD91 ligand-antigenic molecule complexes formed can be washed free of unbound antigenic molecules prior to eluting the CD91 ligand-antigenic molecule complexes off the solid phase. The association of the antigenic molecules with the CD91 ligands can be assayed by any method known in the art.

The following example describes a preferred method for preparing non-covalent immunogenic complexes of CD91 ligands and antigenic molecules:
CD91 ligands are prepared, as described in Section 4.2, above. The antigenic molecules (100 μg) and the pretreated CD91 ligand (1 μg-1 mg) are mixed together to give an approximately 5:1 antigenic molecule: CD91 ligand molar ratio. The mixture is then incubated for 15 minutes to 3 hours at 4° to 45° C. in a suitable binding buffer such as one containing 20 mM sodium phosphate, pH 7.2, 350 mM NaCl, 3 mM MgCl₂, 1 nM phenyl methyl sulfonyl fluoride (PMSF), and 1-10 mM ADP. The preparations are centrifuged through a Centricon 10 assembly (Millipore) to remove any unbound peptide. If the CD91 ligand is bound to a solid phase, the CD91 ligand-antigenic molecule complexes formed can be washed free of unbound antigenic molecules prior to eluting the CD91 ligand-antigenic molecule complexes off the solid phase. The association of the antigenic molecules with the CD91 ligands can be assayed by SDS-PAGE.

Following complexing, the immunogenic CD91 ligand-antigenic molecule complexes can optionally be assayed in vivo or in vitro using, for example, the methods described in Section 4.7, below.

In another embodiment of the invention, a linker molecule may be used to non-covalently tether a CD91 ligand to an antigenic molecule. A linker molecule is a bispecific molecule in which one moiety of the linker molecule binds a CD91 ligand, while another moiety of the linker molecule binds an antigenic molecule. Such linker molecules are described in PCT WO 01/8772, which is incorporated by reference herein. In a particular embodiment, the linker molecule is covalently linked to an antigenic molecule and non-covalently linked to a CD91 ligand. In another embodiment, the linker molecule is non-covalently linked to an antigenic molecule and non-covalently linked to a CD91 ligand. In yet another embodiment, the linker molecule is non-covalently linked to an antigenic molecule and covalently linked to a CD91 ligand.

4.4.2 Formation of Covalent CD91 Ligand-Antigenic Molecule Complexes

As an alternative to non-covalent complexes, antigenic molecules covalently attached to CD91 ligands may be used as vaccines to elicit an immune response.

To prepare such covalent CD91 ligand-antigenic molecule complexes, CD91 ligands and antigenic molecules are prepared, as described in Sections 4.2 and 4.1, respectively. The CD91 ligand and the antigenic molecule are then covalently coupled using any method known in the art.

In one embodiment, CD91 ligands may be covalently coupled to antigenic molecules by chemical cross-linking. Chemical cross-linking agents, such as aldehydes, ketones and glyoxals, and their use are well known in the art and are suitable for use in the invention. Cross-lining agents, including but not limited to, protein A, glutaraldehyde, carbodiimide, N-succinimidyl-S-acetyl-thioacetate (SATA), N-succinidyl-3-2-pyridyldithio)propionate (SPDP), and sulfosuccinimidyl 4-(N-maleimidomethyl)cyclohexane-1-carboxylate (sSMCC) can be used. For example, in one embodiment of the invention, glutaraldehyde may be used to cross-ink a CD91 ligand and an antigenic molecule. In addition, other methods for covalent attachment of proteins known in the art can be used in the invention, such as photo cross-linking (See Current Protocols in Molecular Biology, Ausubel et al. (eds.), Greene Publishing Associates and Wiley Interscience, New York and Binder et al., 2000, Nature Immunol. 1:151-155, which are incorporated by reference herein).

4.5 CD91 Ligand-Antigenic Molecule Fusion Proteins

In another embodiment, recombinant fusion proteins, comprising a CD91 ligand fused via a peptide bond to an antigenic protein, may be used to treat or prevent cancer or an infectious disease. To produce such a recombinant fusion protein, an expression vector is constructed using nucleic acid sequences encoding a CD91 ligand fused to sequences encoding an antigenic protein, using recombinant methods known in the art, such as those described in Section 4.1.3, above.

CD91 ligand-antigenic molecule fusions are then expressed and isolated. In one embodiment, the N-terminal portion of a CD91 ligand or CD91 ligand fragment is fused to the C-terminal portion of an antigenic molecule. In another embodiment, the C-terminal portion of a CD91 ligand or CD91 ligand fragment is fused to the N-terminal portion of an antigenic molecule. Such fusion proteins can be used to elicit an immune response (Suzue et al., 1997, Proc. Natl. Acad. Sci. U.S.A. 94:13146-51). By specifically designing the antigenic protein portion of the molecule, such fusion proteins can be used to induce an immune response and in immunotherapy against target diseases or disorders.

4.5.1 Recombinant Expression and Production of CD91 Ligand-Antigenic Molecule Fusion Proteins

To produce CD91 ligand-antigenic molecule fusion proteins, a nucleotide sequence encoding a CD91 ligand or a CD91 ligand fragment and an antigenic molecule can be introduced into cells including, but not limited to, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. The choice of cell type depends on the type of antigenic protein being expressed, and can be determined by one of skill in the art

Preferably, the cells used in the methods of the invention are of mammalian origin. Mammals contemplated by this aspect of the invention include humans, companion animals (e.g., dogs and cats), livestock animals (e.g., sheep, cattle, goats, pigs and horses), laboratory animals (e.g., mice, rats and rabbits), and captive or free wild animals.

In various embodiments, any cells, preferably human cells, can be used in the present methods for producing CD91 ligand-antigenic protein fusion proteins. Introduction of gene constructs encoding the CD91 ligand and the antigenic molecule can be carried out by any method known in the art, including gene therapy art, such as but not limited to transfection, electroporation, microinjection, infection with a viral or bacteriophage vector containing the nucleic acid sequence encoding the CD91 ligand and antigenic protein, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29: 69-92) may be used in accordance with the present invention, provided that the necessary developmental and physiological functions of the recipient cells are not disrupted. The technique should provide for the stable transfer of the nucleic acid sequence encoding the CD91 ligand and antigenic protein to the cell, so that the sequence is expressible and preferably heritable and expressible by its cell progeny.

In addition, the methods for recombinant production described in section 4.1.3 and its subsections hereinabove can be used.

4.6 Therapeutic and Prophylactic Uses of CD91 Ligand-Antigenic Molecule Complexes and Fusion Proteins

The present invention encompasses methods for treatment and prevention of cancer and other diseases using complexes or fusion proteins of CD91 ligands and antigenic molecules. The present invention also provides methods of inducing an immune response to an antigenic molecule using the complexes and fusion proteins of the invention.

In one embodiment, an immunogenic amount of complex or fusion protein of a CD91 ligand and an antigenic molecule, wherein the antigenic molecule displays the antigenicity of an antigen that causes or is associated with a disease, can be used in the treatment or prevention of the disease. In a specific embodiment, the CD91 ligand and the antigenic molecule of the complex are noncovalently linked. In another embodiment, the CD91 ligand and the antigenic molecule of the complex are chemically cross-linked

In a preferred aspect of the invention, the purified CD91 ligand-antigenic molecule complex or fusion protein vaccines may have particular utility in the treatment of human cancer or other diseases. It is appreciated, however, that the vaccines developed using the principles described herein will be useful in treating diseases of other mammals, for example, farm animals including cattle, horses, sheep, goats, and pigs, and household pets including cats and dogs.

The invention also provides a method of treating a disease or disorder amenable to treatment by induction of an immune response against an antigenic molecule comprising administering to a subject having the disease or disorder an immunogenic amount of a purified complex comprising the antigenic molecule noncovalently bound to, or cross-linked to, a CD91 ligand.

The invention also provides a method of treating a disease or disorder amenable to treatment by induction of an immune response against an antigenic molecule comprising administering to a subject having the disease or disorder an immunogenic amount of a purified fusion protein comprising the antigenic molecule fused via a peptide bond to a CD91 ligand.

4.6.1 Inducing an Immune Response to an Antigen

The complexes and fusion proteins of the invention can be used to induce an immune response to an antigenic molecule in a subject.

Without being bound by any particular theory, it is believed that an immune response to an antigenic molecule is induced by the complexes and fusion proteins of the invention by enhancing the uptake of the antigenic molecule by antigen presenting cells, including macrophages, dendritic cells and B cells.

4.6.2 Passive Immunotherapy

CD91 ligand-antigenic molecule complexes and fusion proteins can also be used for passive immunotherapy against cancer or other diseases. Passive immunity is the short-term protection of a host, achieved by the administration of pre-formed antibody (e.g., in purified form or by administering serum containing a pre-formed antibody(ies)), directed against an antigen or pathogenic organism of interest, e.g., a tumor or viral antigen. For example, CD91 ligand-antigenic molecule complexes or fusion proteins may be used to elicit an immune response in a subject, the sera removed from the subject and used for treatment or prevention of a disease in a subject having a disease caused by the presence of a common antigen.

4.7 Determination of Immunogenicity of CD91 Ligand-Antigenic Molecule Complexes and Fusion Proteins

Optionally, CD91 ligand-antigenic molecule complexes and fusion proteins can be assayed for immunogenicity using any method known in the art. By way of example but not limitation, one of the following three procedures can be used.

4.7.1 MLTC Assay

Briefly, mice are injected with the CD91 ligand-antigenic molecule complex or fusion protein, using any convenient route of administration. As a negative control, other mice are injected with CD91 ligand-antigenic molecule complexes or fusion proteins not associated with the antigen of interest, or cells containing CD91 ligand-antigenic molecule complexes or fusion proteins not associated with the antigen of interest. Cells containing the antigen of interest may act as a positive control for the assay. The mice are injected twice, 7-10 days apart. Ten days after the last immunization, the spleens are removed and the lymphocytes released. The released lymphocytes may be re-stimulated subsequently in vitro by the addition of dead cells that expressed the antigen of interest.

For example, 8×10⁶ immune spleen cells may be stimulated with 4×10⁴ mitomycin C treated or γ-irradiated (5-10,000 rads) cells containing the antigen of interest (or cells transfected with an appropriate gene, as the case may be) in 3 ml RPMI medium containing 10% fetal calf serum. In certain cases 33% secondary mixed lymphocyte culture supernatant may be included in the culture medium as a source of T cell growth factors (See, Glasebrook, et al., 1980, J. Exp. Med. 151:876). To test the primary cytotoxic T cell response after immunization, spleen cells may be cultured without stimulation. In some experiments spleen cells of the immunized mice may also be re-stimulated with antigenically distinct cells, to determine the specificity of the cytotoxic T cell response.

Six days later the cultures are tested for cytotoxicity in a 4 hour ⁵¹Cr-release assay (see Palladino et al., 1987, Cancer Res. 47:5074-5079 and Blachere, et al., 1993, J. Immunotherapy 14:352-356). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 20 mCi ⁵¹Cr/ml for one hour at 37° C. The cells are washed three times following labeling. Each assay point (E:T ratio) is performed in triplicate and the appropriate controls incorporated to measure spontaneous ⁵Cr release (no lymphocytes added to assay) and 100% release (cells lysed with detergent). After incubating the cell mixtures for 4 hours, the cells are pelletted by centriflgation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

4.7.2 CD4+T Cell Proliferation Assay

Primary T cells are obtained from spleen, fresh blood, or CSF and purified by centriflgation using FICOLL-PAQUE PLUS (Pharmacia, Upsalla, Sweden) essentially as described by Kruse and Sebald, 1992, EMBO J. 11: 3237-3244. The peripheral blood mononuclear cells are incubated for 7-10 days with a lysate of cells expressing an antigen of interest Antigen presenting cells may, optionally be added to the culture 24 to 48 hours prior to the assay, in order to process and present the antigen in the lysate. The cells are then harvested by centrifugation, and washed in RPMI 1640 media (GibcoBRL, Gaithersburg, Md.). 5×10⁴ activated T cellsvell (PHA-blasts) are in RPMI 1640 media containing 10% fetal bovine serum, 10 mM HEPES, pH 7.5,2 mM LIglutamine, 100 units/ml penicillin G, and 100 μg/ml streptomycin sulphate in 96 well plates for 72 hrs at 37° C., pulsed with 1 μCi ³H-tymidine (DuPont ENi, Boston, Mass.)/well for 6 hrs, harvested, and radioactivity measured in a TOPCOUNT scintillation counter Packard Instrument Co., Meriden, Conn.).

4.7.3 Antibody Response Assay

In one embodiment of the invention, the immunogenicity of a CD91 ligand-antigenic molecule complex or fusion protein is determined by measuring antibodies produced in response to the vaccination with the complex or fusion protein, by an antibody response assay, such as an enzyme-linked immunosorbent assay (ELISA) assay. Methods for such assays are well known in the art (see, e.g., Section 2.1 of Current Protocols in Immunology, Coligan et al. (eds.), John Wiley and Sons, Inc. 1997). In one mode of the embodiment, microtitre plates (96-well Immuno Plate II, Nunc) are coated with 50 μl/well of a 0.75 μg/ml solution of a purified, non-CD91 ligand-complexed form of the antigen used in the vaccine (e.g. Aβ42) in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The anti-antigenic molecule antibody activity is then measured calorimetrically after incubating at 20° C. for 1 hour with 50 μl/well of sheep anti-mouse or anti-human immunoglobulin, as appropriate, conjugated with horseradish peroxidase (Amersham) diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a Kontron SLT-210 photometer (SLT Lab-instr., Zurich, Switzerland) at 492 um (ref 620 En).

4.8 Dosage Regimens

For treatment of cancer, dosages of CD91 ligand-antigenic molecule complexes and fusion proteins may be extrapolated from prior art methods established in experimental tumor models (Blachere et al., 1993, J. Immunotherapy 14:352-356). For other diseases, dosages may be extrapolated from prior art methods established in an appropriate experimental disease model. Extrapolation to human dosages is based on body weight and surface area. For example, prior art methods of extrapolating human dosage based on body weight can be carried out as follows: since the conversion factor for converting the mouse dosage to human dosage is Dose Human per kg=Dose Mouse per kg×12 (See Freireich, E. J., et al., 1966, Cancer Chemotherap. Rep. 50:219-244), the effective dose of CD91 ligand-antigenic molecule complexes or fusion proteins in humans weighing 70 kg should be 1 mg/kg+12×70, i.e., about 6 mg (5.8 mg).

Drug doses are also given in milligrams per square meter of body surface area because this method rather than body weight achieves a good correlation to certain metabolic and excretionary functions (Shirkey, 1965, JAMA 193:443). Moreover, body surface area can be used as a common denominator for drug dosage in adults and children as well as in different animal species as indicated below in Table 1 (Freireich et al., 1966, Cancer Chemotherap. Rep. 50:219-244).

TABLE 1
REPRESENTATIVE SURFACE AREA TO WEIGHT
RATIOS (km) FOR VARIOUS SPECIES¹

| | Body Weight | Surface Area | |
|---|---|---|---|
| Species | (kg) | (Sqm) | km Factor |
| Mouse | 0.02 | 0.0066 | 3.0 |
| Rat | 0.15 | 0.025 | 5.9 |
| Monkey | 3.0 | 0.24 | 12 |
| Dog | 8.0 | 0.40 | 20 |
| Human, Child | 20 | 0.80 | 25 |
| Adult | 60 | 1.6 | 37 |

¹Freireich, et al., 1966, Cancer Chemotherap. Rep. 50: 219-244.

Example: To express a mg/kg dose in any given species as the equivalent mg/sq m dose, multiply the dose by the appropriate km factor. In adult human, 100 mg/kg is equivalent to 100 mg/kg×37 kg/sq m=3700 mg/sq m.

As an alternative to the standard extrapolation, the dosage range for the complexes and fusion proteins of the invention is 1 μg to 5 mg, 1 μg to 1 mg, or 5 μg to 500 μg, with a preferred range being 10 μg to 250 μg. Representative dosages include 10 μg, 25 μg, 50 μg, 75 μg, 100 μg, 150 μg, 200 μg and 250 μg.

The doses recited above are preferably given once weekly for a period of about 4-6 weeks, and the mode or site of administration is preferably varied with each administration. In a preferred example, intradermal administrations are given, with each site of administration varied sequentially. Thus, by way of example and not limitation, the first injection may be given subcutaneously on the left arm, the second on the right arm, the third on the left belly, the fourth on the right belly, the fifth on the left thigh, the sixth on the right thigh, etc. The same site may be repeated after a gap of one or more injections. Also, split injections may be given. Thus, for example, half the dose may be given in one site and the other half on an other site on the same day.

Alternatively, the mode of administration is sequentially varied, e.g. weekly injections are given in sequence intradermally, intramuscularly, intravenously or intraperitoneally.

After 4-6 weeks, further injections are preferably given at two-week intervals over a period of time of one month. Later injections may be given monthly. The pace of later injections may be modified, depending upon the patient's clinical progress and responsiveness to the immunotherapy.

For prevention, the subject is immunized by administration of the vaccine formulation, in at least one dose, and preferably two or more doses. However, the subject may be administered as many doses as is required to maintain a state of immunity. For example, boosters given at regular intervals, i.e., at six months or yearly, may be desirable in order to sustain immunity at an effective level.

4.9 Vaccine Formulation

Complexes and fusion proteins of CD91 ligands and antigenic molecules purified by the methods of the invention may be formulated into pharmaceutical preparations (vaccines) for administration to mammals for treatment or prevention of cancer or other diseases. Drug solubility and the site of absorption are factors which should be considered when choosing the route of administration of a therapeutic agent. CD91 ligand-antigenic molecule complexes of the invention may be administered using any desired route of administration, including but not limited to, e.g., intradermally, subcutaneously, intravenously or intramuscularly, although intradermally is preferred. Advantages of intradermal administration include use of lower doses and rapid absorption, respectively. Mucosal routes of administration include, but are not limited to, oral, rectal and nasal administration. Preparations for mucosal administrations are suitable in various formulations as described below. The route of administration can be varied during a course of treatment.

The invention also contemplates pharmaceutical compositions (vaccines) comprising a CD91 ligand-antigenic molecule complex or fusion protein and an adjuvant. The complex or fusion protein is preferably purified. More than one adjuvant can be used in pharmaceutical compositions of the invention. Some adjuvants that may be used in the invention include, but are not limited to: heat shock proteins, saponin adjuvants (e.g., QS-21), alpha 2 macroglobulin, lipopolysaccharide (LPS), immunostimulatory oligonucleotides which include CpG oligonucleotides, and complexes of heat shock proteins and antigenic molecules, such as peptides, or the like. Furthermore, the following patents and printed publications disclose immunostimulatory oligonucleotides which include CpG oligonucleotides that can be used in the compositions of the invention: U.S. Pat. Nos. 6,207,646; 6,339,068; 6,239,116; 6,429,199; and PCT Patent publication, WO 01/22972, WO 00/06588, by Krieg et al.; WO 01/83503; WO 01/55370; and WO 01/12804 by Agrawal; WO 02/052002 by Fearon et al.; WO 01/35991 by Tuck et al.; WO 01/12223 by Van Nest; WO 98/55495; WO 99/62923 by Schwartz; U.S. Pat. No. 6,406,705 by Davis et al.; and PCT Patent publication WO 02/26757 by Kandimalla et al., all of the forgoing are incorporated herein by reference in their entireties. In a preferred embodiment, a combination of saponin adjuvants (e.g., QS-21) and immunostimulatory oligonucleotides (e.g., a CpG oligonucleotide) are used.

Other suitable adjuvants that can be used in the invention can be found in A Compendium of Vaccine Adjuvants and Excipients (2^{nd} Edition), Vogel, F., Powell, M., and Alving, C., in Vaccine Design—The Subunit and Adjuvant Approach, Powell, M., Newman, M., Burdman, J., Editors, Plenum Press, New York, 1995, pp. 141-227, and 2^{nd} Meeting on Novel Adjuvants Currently In/Close to Human Clinical Testing, World Health Organization—Organization Mondiale de la Sante Foundation Merieux, Annecy, France, 5-7 Jun. 2000, Kenney, Ill., Rabinovich, N. R., Pichyangkul, S., Price, V., and Engers, H., Vaccine, 20 (2002) 2155-63. All of which are incorporated herein by reference.

In a specific embodiment, a complex or fusion protein of the invention is administered separately from one or more adjuvants. In one embodiment, a complex or fusion protein of the invention and an adjuvant are administered in a sequence and within a time interval such that the complex or fusion protein and an adjuvant can act together to provide an increased benefit that is greater than either administered alone. In a specific embodiment, the complex or fusion protein and an adjuvant are administered sufficiently close in time so as to provide the desired therapeutic or prophylactic outcome. Each can be administered simultaneously and/or separately, in any appropriate form and by any suitable route. In one embodiment, the complex or fusion protein and an adjuvant are administered by different routes of administration. In an alternate embodiment, each is administered by the same route of administration The complex or fusion protein and an adjuvant can be administered at the same or different sites, e.g. arm and leg.

In specific embodiments, a complex or fusion protein of the invention and an adjuvant are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other specific embodiments, a complex or fusion protein of the invention and an adjuvant are administered 2 to 4 days apart, 4 to 6 days apart, 1 week a part, 1 to 2 weeks apart, 2 to 4 weeks apart, one moth apart, 1 to 2 months apart, or 2 or more months apart. In preferred embodiments, a complex or fusion protein of the invention and an adjuvant are administered in a time frame where both are still active. One skilled in the art would be able to determine such a time frame by determining the half life of each administered component.

Compositions comprising CD91 ligand-antigenic molecule complexes or fusion proteins formulated in a compatible pharmaceutical carrier may be prepared, packaged, and labeled for treatment of the indicated cancer or infectious disease. In preferred aspects, an amount of CD91 ligand-antigenic molecule complex or fusion protein is administered to a human that is in the range of about 2 to 150 μg, preferably 20 to 200 μg, most preferably about 5 μg, given once weekly for about 4-6 weeks, intradermally with the site of administration varied sequentially.

If the complex or fusion protein is water-soluble, then it may be formulated in an appropriate buffer, for example, phosphate buffered saline or other physiologically compatible solutions. Alternatively, if the resulting complex has poor solubility in aqueous solvents, then it may be formulated with a non-ionic surfactant such as Tween, or polyethylene glycol. Thus, the CD91 ligand-antigenic molecule complexes and fusion proteins and their physiologically acceptable solvates may be formulated for administration by inhalation or insufflation (either through the mouth or the nose) or oral, buccal, parenteral, rectal administration.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oiL oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the complexes. Such compositions may take the form of tablets or lozenges formulated in conventional manner.

In a specific embodiment, the complexes and compositions of the present invention are administered intrathecally by an implant be placed in or near the lesioned area Suitable implants include, but are not limited to, gelfoam, wax, liposome or mnicroparticle-based implants. Such compositions are preferably used when it is desired to achieve sustained release of the CD91 ligand-antigenic molecule complexes.

For administration by inhalation, the complexes and fusion proteins may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the complexes and a suitable powder base such as lactose or starch.

The complexes and fusion proteins may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use.

The complexes and fusion proteins may also be formulated in rectal compositions such as suppositories or retention enemas, e.g., containing conventional suppository bases such as cocoa butter or other glycerides.

In addition to the formulations described previously, the complexes and fusion proteins may also be formulated as a depot preparation. Such long acting formulations may be administered by implantation (for example, subcutaneously or intramuscularly) or by intramuscular injection. Thus, for example, the complexes may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

The invention also provides kits for carrying out the therapeutic regimens of the invention. Such kits comprise in one or more containers therapeutically or prophylactically effective amounts of CD91 ligand-antigenic molecule complexes or fusion proteins in pharmaceutically acceptable form (preferably purified), optionally with a pharmaceutically acceptable carrier. The kit optionally further comprises in the same or different container an adjuvant, e.g., a saponin. The CD91 ligand-antigenic molecule complexes or fusion proteins in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex or fusion protein may be lyophilized or desiccated; in this instance, the kit optionally further comprises in a container apharmaceutically acceptable solution (e.g., saline, dextrose solution, etc.), preferably sterile, to reconstitute the complex or fusion protein to form a solution for injection purposes. Kits directed to the treatment or prevention of cancer or infectious disease optionally further comprise in a separate container, antigen presenting cells (APCs), which may be sensitized. If the APCs are not sensitized, the kit may further comprise a purified antigenic molecule for sensitizing the APCs. The APCs are preferably purified. The invention also contemplates kits which, in addition to the components above, also include one or more therapeutic or preventive compositions known in the art in the treatment or prevention of a disease or disorder. Examples of such compositions, listed by way of example and not limitation are found in section 4.12, below.

In another embodiment, a kit of the invention further comprises a needle or syringe, preferably packaged in sterile form, for injecting the complex or fusion protein, and/or a packaged alcohol pad. Instructions are optionally included for administration of CD91 ligand-antigenic molecule complexes or fusion proteins by a clinician or by the patient.

4.10 Determination of Vaccine Efficacy

The immunopotency of the complexes and fusion proteins of the invention can be determined by monitoring the immune response in test animals following immunization with the complexes and fusion proteins of the invention, or by use of any immunoassay known in the art. Generation of a humoral (antibody) response and/or cell-mediated immunity, may be taken as an indication of an immune response. Test animals may include mice, hamsters, dogs, cats, monkeys, rabbits, chimpanzees, etc., and eventually human subjects.

Methods of introducing the vaccine may include oral, intracerebral, intradermal, transdermal, intramuscular, intraperitoneal, intravenous, subcutaneous, intranasal or any other standard routes of immunization. The immune response of the test subjects can be analyzed by various approaches such as: the reactivity of the resultant immune serum to the antigen of interest, as assayed by known techniques, e.g., immunosorbant assay (BLISA), immunoblots, radioimnunoprecipitations, etc., or by protection of the immunized host against cancer or the infectious disease.

As one example of suitable animal testing of a vaccine protective a disease, the vaccine of the invention may be tested in rabbits for the ability to induce an antibody response to the antigenic molecule. Male specific-pathogen-free (SPF) young adult New Zealand White rabbits maybe used. The test group each receives a fixed concentration of the vaccine. A control group receives an injection of 1 mM Tris-HCl pH 9.0 without the antigen molecule.

Blood samples may be drawn from the rabbits every one or two weeks, and serum analyzed for antibodies to the antigenic molecule. The presence of antibodies specific for the antigen may be assayed, e.g., using an ELISA assay.

4.11 Monitoring of Effects During Immunotherapy

Optionally, the effect of immunotherapy with CD91 ligand-antigenic molecule complexes or fusion proteins on progression of a disease can be monitored by any methods known to one skilled in the art. In addition, cellular immunity may optionally be monitored by methods including but not limited to measuring: a) delayed hypersensitivity as an assessment of cellular immunity, b) activity of cytolytic T-lymphocytes in vitro; c) levels of antigenic molecule.

Delayed hypersensitivity skin tests are also of great value in the overall immunocompetence and cellular immunity to an antigen. Inability to react to a battery of common skin antigens is termed anergy (Sato et al., 1995, Clin. Immunol. Pathol. 74:35-43).

Proper technique of skin testing requires that the antigens be stored sterile at 4° C., protected from light and reconstituted shortly before use. A 25- or 27-gauge needle ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

In another optional method, the activity of cytolytic T-lymphocytes can be assessed in vitro using the following method. Eight×10⁶ peripheral blood-derived T lymphocytes isolated by the Ficoll-Hypaque centrifugation gradient technique, are restimulated with 4×10⁴ mitomycinC-treated cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator cells. In other experiments, T cells are restimulated with antigenically distinct cells. After six days, the cultures are tested for cytotoxicity in a 4 hour ⁵¹Cr-release assay. The spontaneous ⁵¹Cr-release of the targets should reach a level less than 20%. For the anti-MHC class I blocking activity, a tenfold concentrated supernatant of W6/32 hybridoma is added to the test at a final concentration of 12.5% (Heike et al., J. Immunotherapy 15: 165-174).

In immunization procedures, the amount of immunogen to be used and the immunization schedule will be determined by a physician skilled in the art and will be administered by reference to the immune response and antibody titers of the subject.

4.12 Combination Therapy

The complexes and fusion proteins of the invention can be used in combination with other therapeutic compositions known in the art in the treatment or prevention of a disease or disorder, such as immunotherapeutic agents, antineoplastic agents, anti-viral agents, anti-fungal agents, antibiotics and anti-inflammatory agents. The complexes or fusion proteins may be coadministered with one or more therapeutic compositions known in the art or may be administered after or before one or more therapeutic compositions known in the art The invention contemplates combinations of the complexes or fusion proteins of the invention and one or more therapeutic compositions know in the art to be useful in treating or preventing any of the diseases or disorders listed in sections 4.13-4.18 below. The administration of the complexes and fusion proteins of the invention can augment the effect of anti-cancer agents or anti-infectives, and vice versa With combination therapy, additive potency or additive therapeutic effects can be observed. In combinations where synergistic outcomes are achieved (where the therapeutic efficacy is greater than additive), dosages that alone would be suboptimal, can be employed. The use of combination therapy can also provide better therapeutic profiles than the administration of the treatment modality, or the complexes and fusion proteins of the invention alone. Additive or synergistic effects may allow the dosage and/or dosing frequency of either or both modalities to be adjusted to reduce or avoid unwanted or adverse effects. The invention also encompasses pharmaceutical compositions comprising a CD91 ligand-antigenic molecule complex or fusion protein and one or more therapeutic compositions know in the art to be useful in treating or preventing any of the diseases or disorders listed in sections 4.13-4.18 below. The invention also encompasses pharmaceutical compositions comprising an CD91 ligand-antigenic molecule complex or fusion protein and a reduced dose of one or more therapeutic compositions know in the art to be useful in treating or preventing any of the diseases or disorders listed in sections 4.13-4.18 below. As used herein, the phrase “reduced dose” refers to a dose that is below the normally administered range, ie., below the standard dose as suggested by the Physicians'Desk Reference, 54^{th} Edition (2000) or a similar reference.

For example, the complexes or fusion proteins of the invention may be administered in combination with one or more chemotherapeutic agent. Such chemotherapeutic agents are known in the art and include but are not limited to: methotrexate, taxol mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, Gleevecdm (imatinib mesylate), herbimycin A, genistein, erbstatin, and lavendustin A

In other embodiments, suitable chemotherapeutics include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbizine, topotecan, nitrogen mustards, cytoxan, etoposide, 5-fluorouracil, BCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. Moreover, the anti-cancer agent can be, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum containing compounds: | Cisplatin |
| | Carboplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxol |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-γ |
| | Tumor necrosis factor |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein |
| | (IP-10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | tetrathiomolybdate |
| | thalidomide |
| | Thrombospondin-1 (TSP-1) |
| | TNP-470 |
| | Transforming growth factor-beta |
| | (TGF-b) |
| | Vasculostatin |
| | Vasostatin (calreticulin fragment) |
| | ZD6126 |
| | ZD 6474 |
| | farnesyl transferase inhibitors |
| | (FTI) |
| | bisphosphonates |
| Antimitotic agents: | allocolchicine |
| | Halichondrin B |
| | colchicine |
| | colchicine derivative |
| | dolstatin 10 |
| | maytansine |
| | rhizoxin |
| | thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Additional anti-cancer agents that may be used in the methods and compositions of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofuiin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs that can be used include, but are not limited to: 20epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevuiinic acid; amnubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicohn glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-poiphyrin; cladribine; clomifene analogues; clotnmazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocannycin SA; ebselen; ecomustine; edelfosine; edrecolomab; eflornithine; clemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ihmofosine; ilomastat; imidazoacridones; imiquimod; immunostimulant peptides; insulin-like growth factor-1 receptor inhibitor, interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor, multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytiol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhlbitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargratnostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; ssta; suramin; swainsonine; synthetic glycosaminoglycans; tallimustine; tamoxifen methiodide; tauromustine; tazarotene; tecogalan sodium; tegafur, tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trinmetrexate; triptorelin; tropisetron; turosteride; tyrosine kinase inhibitors; tyrphostins; UTBC inhibitors; ubenimex; urogenital sinus-derived growth inhibitory factor; urolinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The complexes or fusion proteins of the invention may also be administered in combination with one or more cytokines or immunomodulatory agents. In various embodiments, the cytokine is selected from the group consisting of IL-1α, IL-1β, IL-2, IL-3, IL-4, IL-5, IL-6, IL7, IL-8, IL-9, IL-10, IL-11, IL-12, IFNα, IFNβ, IFNγ, TNFα, TNFβ, G-CSF, GM-CSF, TGF-β, IL-15, IL-18, GM-CSF, INF-γ, INF-α, SLC, endothelial monocyte activating protein-2 (MAP2), MIP-3α, MP-3β, or an MHC gene, such as HLA-B7. Additionally, other exemplary cytokines include other members of the TNF family, including but not limited to TNF-α-related apoptosis-inducing ligand (TRAEL), TNF-α-related activation-induced cytokine (TRANCE), TNF-α-related weak inducer of apoptosis (TWEAK), CD40 ligand (CD40L), LT-α, LT-β, OX4OL, CD4OL, FasL, CD27L, CD30L, 4-1BBL, APRIL, LIGHT, TL1, TNFSF16, TNFSF17, and AITR-L, or a functional portion thereof. See, e.g., Kwon et al., 1999, Curr. Opin. Immunol. 11:340-345 for a general review of the TNF family.

The complexes or fusion proteins of the invention may also be administered in combination with one or more antifungal agents. Antifungal agents suitable for use in the invention, include but are not limited to: polyenes (e.g. amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofildvin, oligomycins, neomycin undecylenate, pyrrolnitrin, siccanin, tubercidin, and viridin.

Also encompassed by the invention are combination therapies using a CD91 ligand-antigenic molecule complex or fusion protein with an antibiotic agent. Antibiotic agents suitable for use in the invention, include but are not limited to: aminoglycoside antibiotics (e.g., apramycin, arbekacin, bambeimycins, butirosin, dibekacin, neomycin, neomycin, undecylenate, netilmcin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and iripenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefnetazole, and cetlinox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4diaminopyrinidines (e.g., brodimoprim), nitrofurans (e.g., furaltadone, and fuirazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfimide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Also encompassed by the invention are combination therapies using a CD91 ligand-antigenic molecule complex or fusion protein with an antiviral agent. Such antiviral agents include but are not limited to: ribavirin, rifampicin, AZT, ddL, ddC, acyclovir and ganciclovir.

The invention also contemplates the combination of a CD91 ligand-antigenic molecule complex or fusion protein and one or more immunoreactive reagents. Immunoreactive reagents include antibodies, molecules or proteins engineered to include (i.e., comprise) the antigen binding portion of an antibody, molecules or proteins engineered to include an antigen binding domain that mediates antibody dependent immune responses, a peptide or domain that interacts specifically with the antigen of interest, or any antigen binding domain that interacts with an antigen/epitope of interest, and the domain of the constant region of an antibody that mediates antibody dependent immune effector cell responses or processes. Examples of such domains or regions within the Ab constant region that can be used in the present invention include those disclosed in Reddy et al., 2000, J. Immunol. 164(4):1925-33; Coloma et al., 1997, Nat Biotechnol. 15(2):159-63; Carayannopoulos et al., 1994, Proc Natl. Acad. Sci. U.S.A. 91(18):8348-52; Morrison, 1992, Annu Recombinant expression vector Immunol. 10:239-65; Traunecker et al., 1992, Int. J. Cancer Suppl., 7:51-2; Gillies et al., 1990, Hum. Antibodies Hybridomas, 1(1):47-54; each of which is incorporated herein by reference in its entirety. Examples of antibodies useful in the methods and compositions of the invention include, but are not limited to, MDX-010 (Medarex, N.J.) which is a humanized anti-CTLA-4 antibody currently in clinic for the treatment of prostate cancer, SYNAGIS® (MedImmune, Md.) which is a humanized anti-respiratory syncytial virus (RSV) monoclonal antibody for the treatment of patients with RSV infection; HERCEPTIN® (Trastuzumab) (Genentech, Calif.) which is a humanized anti-HER2 monoclonal antibody for the treatment of patients with metastatic breast cancer; REMICADE® (infliximab) (Centocor, Pa.) which is a chimeric anti-TNFα monoclonal antibody for the treatment of patients with Crone's disease; REOPRO® (abeiximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD18 F(ab′)₂ (Genentech); CDP860 which is a humanized anti-CD18 F(ab!)₂ (Celltech, UK); PRO542 which is an anti-HIV gp120 antibody fused with CD4 (Progenics/Genzyme Transgenics); Ostavir which is a human anti Hepatitis B virus antibody (Protein Design Lab/Novartis); PROTOVIR™ which is a humanized anti-CMV IgG1 antibody (Protein Design Lab/Novartis); MAK-195 (SEGARD) which is murine anti-TNF-α F(ab′)₂ (Knoll Pharma/BASF); IC14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); BEC2 which is a murine anti-idiotype (GD3 epitope) IgG antibody (InClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LIDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DIAGNOSTIC REAGENT antibody (Techniclone); ABX-IL8 is a human anti-IL8 antibody (Abgenix); anti-CD11a is a humanized IgG1 antibody (Genentech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is aprimatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-αFab fragment (Celltech); IDEC-151 is a primatized anti-CD4 IgG1 antibody (IDEC Pharm/SmithKline Beecham); MDX-CD4 is a human anti-CD4 IgG antibody (edarex/Eisai/Gemnab); CDP571 is a humanized anti-TNF-αIgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA4 IgG antibody (Elan); MDX-33 is a human anti-CD64 (FcγR) antibody (Medarex/Centeon); SCH55700 is a humanized anti-IL-5 IgG4 antibody (Celltech/Schering); SB-240563 and SB-240683 are humanized anti-IL-5 and IL-4 antibodies, respectively, (SmithKline Beecham); rhuMab-E25 is a humanized anti-IgE IgG1 antibody (Genentech/Norvartis/Tanox Biosystems); ABX-CBL is a murine anti CD-147 IgM antibody (Abgenix); BTI-322 is a rat anti-CD2 IgG antibody (Medimimune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β₂-integrin IgG antibody (LeukoSite); Anti-LFA-1 is a murine anti CD18 F(ab′)₂ (Pasteur-Merieux/Immunotech); CAT-152 is a human anti-TGF-β₂ antibody (Cambridge Ab Tech); and Corsevin M is a chimeric anti-Factor VII antibody (Centocor). Preferred antibodies for use include anti-CTLA-4 antibodies and anti-41BB antibodies. The above-listed immunoreactive reagents, as well as any other immunoreactive reagents, may be administered according to any regimen known to those of skill in the art, including the regimens recommended by the suppliers of the immunoreactive reagents.

4.13 Target Cancers

Types of cancers that can be treated or prevented by the compositions and methods of the present invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrbm's macroglobulinemia, and heavy chain disease.

In a specific embodiment, the cancer is metastatic. In another specific embodiment, the patient having a cancer is immunosuppressed by reason of having undergone anti-cancer therapy (e.g., chemotherapy radiation) prior to administration of the CD91 ligand-antigenic molecule complexes or fusion proteins.

There are many reasons why immunotherapy as provided by the present invention is desired for use in cancer patients. First, if cancer patients are immunosuppressed, surgery with anesthesia and subsequent chemotherapy may worsen the immunosuppression. With appropriate immunotherapy in the preoperative period, this immunosuppression may be prevented or reversed. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

The preventive and therapeutic compositions and methods of the invention are directed at enhancing the immunocompetence of the cancer patient either before surgery, at or after surgery, and to induce tumor-specific immunity to cancer cells, with the objective being inhibition of cancer, and with the ultimate clinical objective being total cancer regression and eradication. The methods of the invention can also be used in individuals at enhanced risk of a particular type of cancer, e.g., due to familial history or environmental risk factors.

4.14 Target Infectious Diseases

Infectious diseases that can be treated or prevented by the compositions and methods of the present invention are caused by infectious agents including, but not limited to, viruses, bacteria, fungi protozoa and parasites. The invention is not limited to treating or preventing infectious diseases caused by intracellular pathogens.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by the methods of the present invention are caused by bacteria including, but not limited to, bacteria that have an intracellular stage in its life cycle, mycobacteria, rickettsia, mycoplasma, neisseria and legionella.

Protozoal diseases that can be treated or prevented by the methods of the present invention are caused by protozoa including, but not limited to, leishmania, kokzidioa, and trypanosoma.

Parasitic diseases that can be treated or prevented by the methods of the present invention are caused by parasites including, but not limited to, chlamydia and rickettsia

4.15 Target Neurodegenteratives Diseases

Neurodegenerative disorders that can be prevented or treated using the compositions and methods of the invention include disorders relating to the central nervous system and/or peripheral nervous system including, but not limited to, cognitive and neurodegenerative disorders such as Alzheimer's Disease, age-related loss of cognitive function and senile dementia, Parkinson's disease, amyotrophic lateral sclerosis, Wilson's Disease, cerebral and progressive supranuclear palsy, Guam disease, Lewy body dementia, prion diseases, such as spongifoim encephalopathies, e.g., Creutzfeldt-Jakob disease, polyglutamine diseases, such as Huntington's disease, myotonic dystrophy, Freidrich's ataxia and other ataxias, well as Gilles de la Tourette's syndrome, seizure disorders such as epilepsy and chronic seizure disorder, stroke, brain or spinal cord trauma, AIDS dementia, alcoholism, autism, retinal ischemia, glaucomna, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as unipolar and bipolar affective disorders. Additional neuropsychiatric and neurodegenerative disorders include, for example, those listed in the American Psychiatric Association's Diagnostic and Statistical manual of Mental Disorders (DSM), the most current version of which is incorporated herein by reference in its entirety.

4.16 Target Endocrine/Metabolic Diseases

Endocrine/metabolic diseases that can be prevented or treated using the compositions and methods of the invention include, but are not limited to, hyperlipidemia, hypercholesterolemia and general disorders of lipid and/or cholesterol metabolism.

4.17 Target Vascular Diseases

Vascular diseases that can be treated or prevented by the compositions and methods of the invention include, but are not limited to, hypertensive heart disease, hypertensive renal disease, secondary hypertension and atherosclerosis.

4.18 Target Autoimmune Diseases

Autoimmune diseases that can be treated or prevented by administration of the antigenic molecules or fusion proteins of the invention include, but are not limited to, insulin-dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A purified, immunogenic complex comprising an antigenic molecule noncovalently bound to a CD91 ligand, and wherein the CD91 ligand is selected from the group consisting of
ApoE, Lipoprotein lipase, Hepatic lipase, Factor IXa, Factor VIIIa, Factor VIIa/TFPL MWP-13, MMP-9, Spingolipid activator protein, PAI-1, Antithrombin III, Tissue factor pathway inhibitor (TFPI), Heparin cofactor II, APP, Thrombospondin-1, Thrombospondin-2, Pseudoinonas exotoxin A, Lactoferin, Rhinovirus and RAP.

2. The complex of claim 1 which is the product of a method comprising noncovalently binding together said CD91 ligand and said antigenic molecule in vitro.

3. A pharmaceutical composition comprising an amount of the purified complex of claim 1 effective for treatment or prevention of a disease or a disorder, and a pharmaceutically acceptable carrier.

4. A composition comprising a plurality of purified complexes of claim 1, each comprising a different antigenic molecule.

5. The composition of claim 4 wherein the antigenic molecule is a protein.

6. A method for preparing immunogenic, in vitro complexes of a CD91 ligand associated with one or more antigenic molecules, said method comprising:
a) incubating a CD91 ligand and one or more antigenic molecules under conditions and for a length of time sufficient for formation of immunogenic complexes of the CD91 ligand noncovalently bound to the antigenic molecules; and
b) isolating said complexes;
wherein the CD91 ligand is selected from the group consisting of ApoE, Lipoprotein lipase, Hepatic lipase, Factor IXa, Factor VIIIa, Factor VIIa/TFPI, MMP-13, MM-9, Spingolipid activator protein, PAI-1, Antithrombin III, Tissue factor pathway inhibitor (TFPI), Heparin cofactor II, APP, Thrombospondin-1, Thrombospondin-2, Pseudomnonas exotoxin A, Lactoferrin, Rhinovirus and RAP.

7. The method of claim 6 wherein the CD91 ligand is purified.

8. A method of treating or preventing cancer or an infectious disease in a subject comprising administering to the subject an amount of a purified complex comprising an antigenic molecule noncovalently bound to a CD91 ligand effective to treat or prevent cancer or an infectious disease, wherein the antigenic molecule displays the antigenicity of a cancer-associated or cancer-specific antigen, or of an antigen of an infectious agent that causes the infectious disease, respectively, and the CD91 ligand is selected from the group consisting of
ApoE, Lipoprotein lipase, Hepatic lipase, Factor IXa, Factor VIIIa, Factor VIIa/TFPI, MMP-13, MMP-9, Spingolipid activator protein, PAI-1, Antithrombin III, Tissue factor pathway inhibitor (TFPI), Heparin cofactor II, APP, Thrombospondin-1, Thrombospondin-2, Pseudomonas exotoxin A, Lactoferrin, Rhinovirus and RAP.

9. A method of inducing an immune response against an antigenic molecule in a subject comprising administering to the subject an immunogenic amount of a purified complex comprising said antigenic molecule noncovalently bound to a CD91 ligand, wherein the CD91 ligand is selected from the group consisting of
ApoE, Lipoprotein lipase, Hepatic lipase, Factor IXa, Factor VIIIa, Factor VIIa/TFPI, MMP-13, MMP-9, Spingolipid activator protein, PAI-1, Antithrombin III, Tissue factor pathway inhibitor (TFPI), Heparin cofactor II, APP, Thrombospondin-1, Thrombospondin-2, Pseudomonas exotoxin A, Lactoferrin, Rhinovirus and RAP, such that an immune response is induced.

10. A method of treating a disease or disorder amenable to treatment by induction of an immune response against an antigenic molecule, comprising administering to a subject having the disease or disorder an immunogenic amount of a purified complex comprising the antigenic molecule noncovalently bound to a CD91 ligand, wherein the CD91 ligand is selected from the group consisting of
ApoE, Lipoprotein lipase, Hepatic lipase, Factor IXa, Factor VIIIa, Factor VIIa/TFPI, MMP-13, MMP-9, Spingolipid activator protein, PAI-1, Antithrombin III, Tissue factor pathway inhibitor (TFPI), Heparin cofactor II, APP, Thrombospondin-1, Thrombospondin-2, Pseudomonas exotoxin A, Lactoferrin, Rhinovirus and RAP.
